(19) United States
(12) Patent Application Publication
(10) Pub. No.: US 2018/0214449 A1
(43) Pub. Date: Aug. 2, 2018

(54) CANCER THERAPY USING JAK INHIBITOR IN COMBINATION WITH MAPK INHIBITORS

(71) Applicant: UBRX, INC., San Diego, CA (US)

(72) Inventors: Hyungsoo KIM, San Diego, CA (US); Ze'ev RONAI, San Diego, CA (US); Keith T. FLAHERTY, Cambridge, MA (US)

(21) Appl. No.: 15/746,206

(22) PCT Filed: Jan. 28, 2016
(86) PCT No.: PCT/US16/15422

§ 371 Date: Jan. 19, 2018

Related U.S. Application Data
(60) Provisional application No. 62/110,290, filed on Jan. 30, 2015.

(51) Int. Cl. A61K 31/519; A61K 31/506; A61K 31/437; A61K 31/44; A61K 35/00

(52) U.S. Cl. CPC A61K 31/519; A61K 31/506; A61K 35/00; A61K 31/44; A61K 31/437

(57) ABSTRACT

Provided herein are new therapeutic regimens for treatment of cancer with a combination of a JAK inhibitor and an inhibitor targeting the MAPK pathway, combinations and methods of use thereof. In some embodiments, described herein are methods for treating cancer in an individual in need thereof, comprising administration of a JAK inhibitor and an inhibitor targeting the MAPK pathway to the individual. In some embodiments, the JAK inhibitor is a JAKI inhibitor.

### CROSS-REFERENCE TO RELATED APPLICATIONS

This application claims the benefit of U.S. Application Ser. No. 62/110,290, filed Jan. 30, 2015, which is hereby incorporated by reference in its entirety.

### BACKGROUND OF THE INVENTION

There is a need for new therapeutic regimens for treatment of cancer.

### SUMMARY OF THE INVENTION

In some embodiments, described herein are methods for treating cancer in an individual in need thereof, comprising administration of a JAK inhibitor and an inhibitor targeting the MAPK pathway to the individual. In some embodiments, the JAK inhibitor is a JAK1 inhibitor. In some embodiments, the JAK inhibitor is a compound selected from

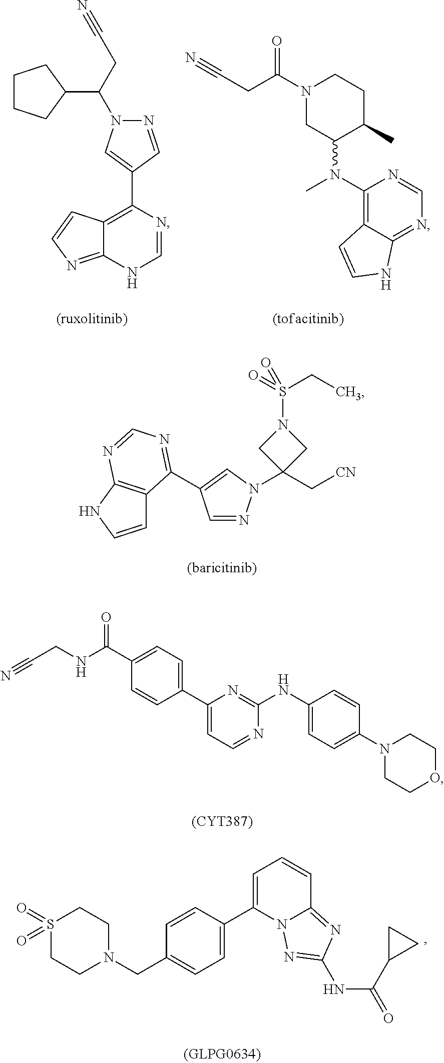 [SMILES: N#CCC(C1CCCC1)n1cc(-c2nc[nH]c3nccc2-3)cn1 ; C[C@@H]1CCN(C(=O)CC#N)CC1N(C)c1ncnc2[nH]ccc12 ; CCS(=O)(=O)N1CC(CC#N)(n2cc(-c3ncnc4[nH]ccc34)cn2)C1 ; N#CCNC(=O)c1ccc(-c2ccnc(Nc3ccc(N4CCOCC4)cc3)n2)cc1 ; "(CYT387)" ; O=C(Nc1nc2cccc(-c3ccc(CN4CCS(=O)(=O)CC4)cc3)n2n1)C1CC1]

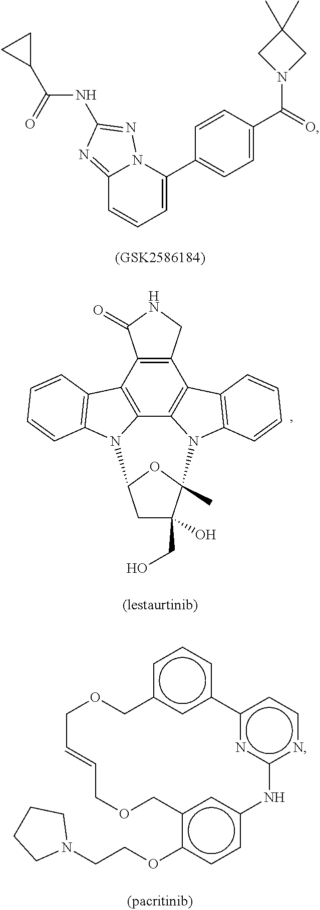

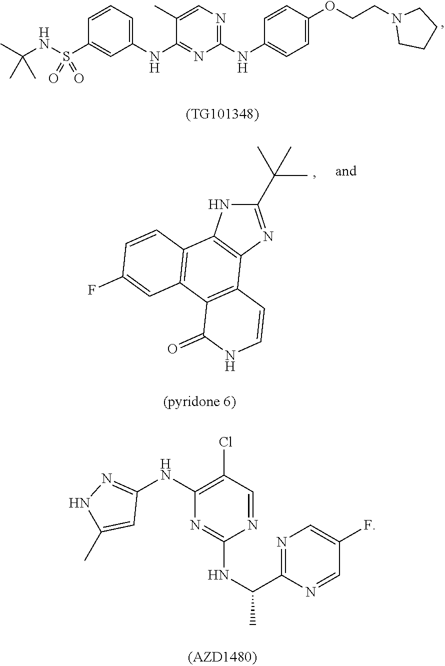 [SMILES: Cc1cnc(Nc2ccc(OCCN3CCCC3)cc2)nc1Nc1cccc(S(=O)(=O)NC(C)(C)C)c1 ; CC(C)(C)c1nc2c3cc[nH]c(=O)c3c3cc(F)ccc3c2[nH]1 ; Cc1cc(Nc2nc(N[C@@H](C)c3ncc(F)cn3)ncc2Cl)n[nH]1]

In some embodiments, the JAK inhibitor is ruxolitinib.

In some embodiments, the inhibitor targeting the MAPK pathway is a B-Raf inhibitor. In some embodiments, the B-Raf inhibitor is selected from

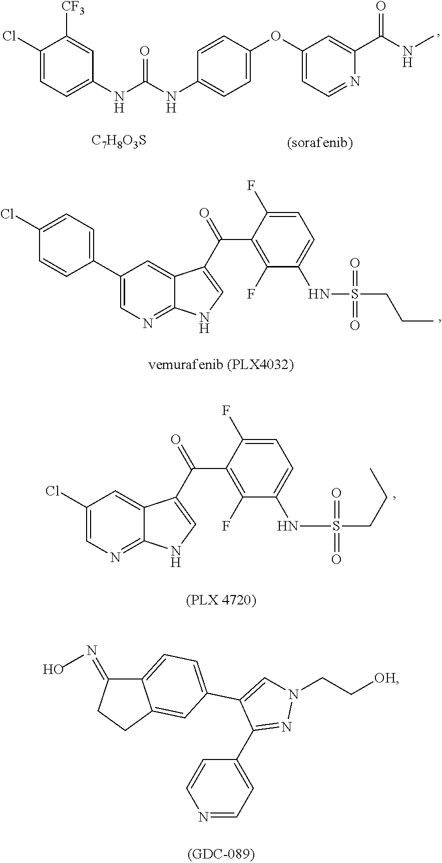

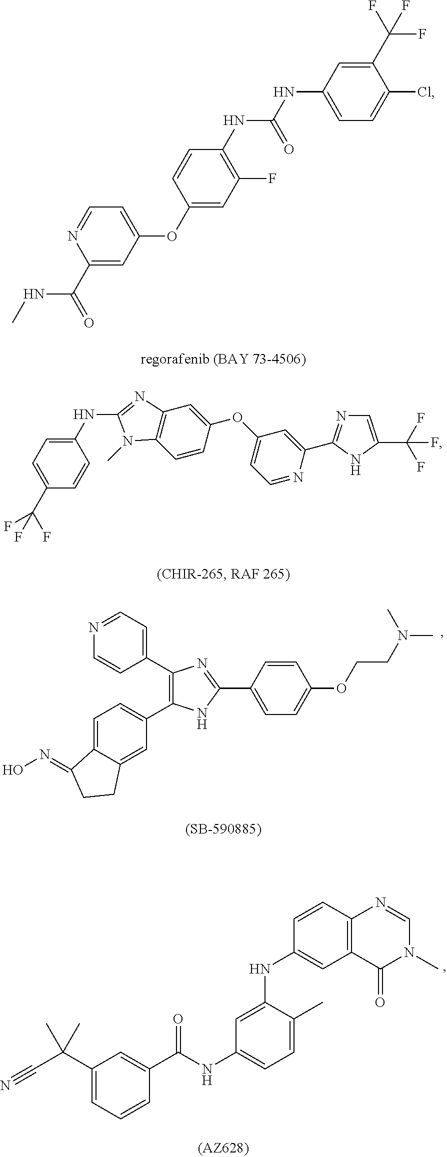 [SMILES: CNC(=O)c1cc(Oc2ccc(NC(=O)Nc3ccc(Cl)c(C(F)(F)F)c3)c(F)c2)ccn1 ; Cn1c(Nc2ccc(C(F)(F)F)cc2)nc2cc(Oc3ccnc(-c4ncc(C(F)(F)F)[nH]4)c3)ccc21 ; CN(C)CCOc1ccc(-c2nc(-c3ccncc3)c(-c3ccc4c(c3)CC/C4=N\O)[nH]2)cc1 ; Cc1ccc(NC(=O)c2cccc(C(C)(C)C#N)c2)cc1Nc1ccc2ncn(C)c(=O)c2c1]

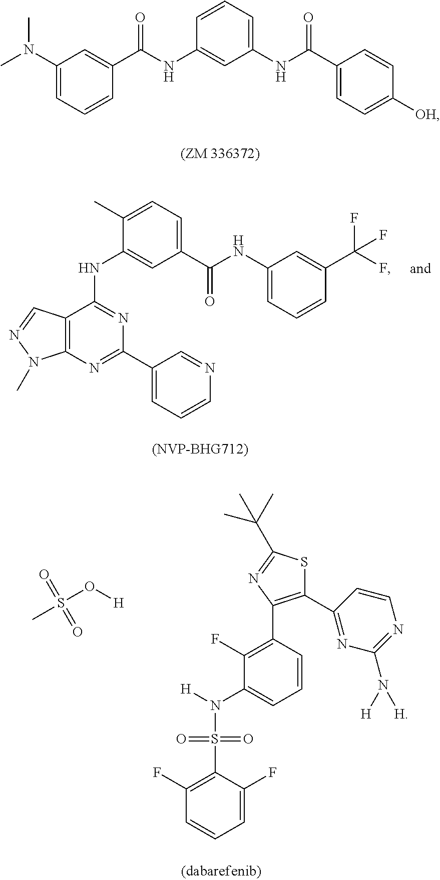

In some embodiments, the B-Raf inhibitor is dabarefenib. In some embodiments, the B-Raf inhibitor is vemurafenib.

In some embodiments, the cancer is a BRAF mutant cancer. In some embodiments, the cancer is selected from the group consisting of head and neck cancer, lung cancer, ovarian cancer, colon cancer, breast cancer, pancreatic cancer, cervical cancer, prostate cancer, and melanoma. In some embodiments, the cancer is melanoma. In some embodiments, the cancer is B-Raf inhibitor resistant melanoma.

In some embodiments, the inhibitor targeting the MAPK pathway is a MEK inhibitor. In some embodiments, the MEK inhibitor is selected from the group consisting of

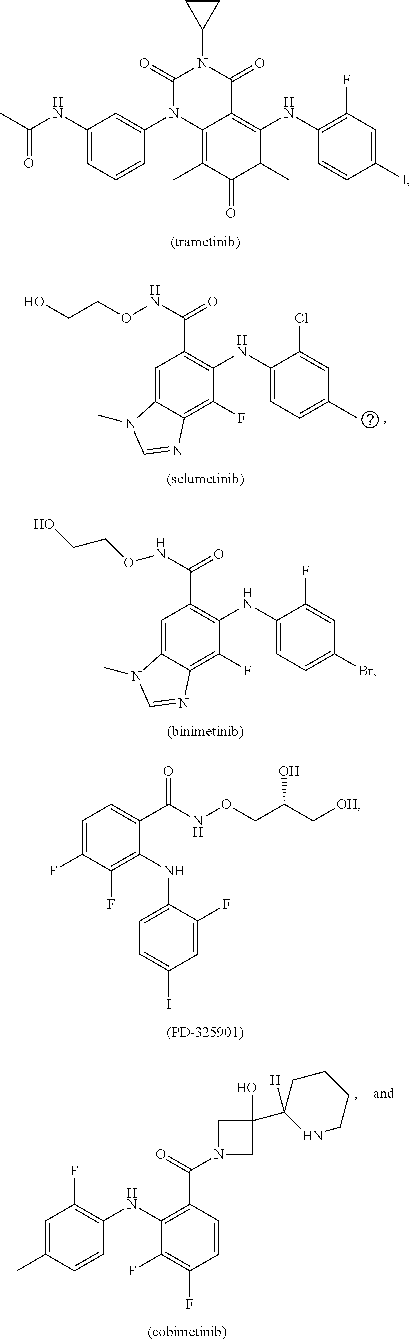

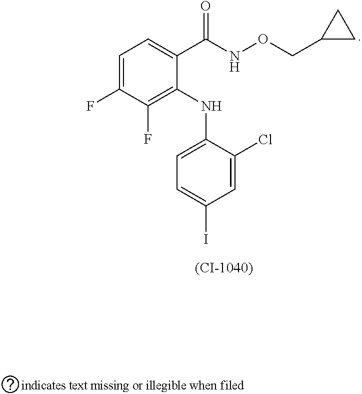 [SMILES: O=C(NOCC1CC1)c1ccc(F)c(F)c1Nc1ccc(I)cc1Cl]

In some embodiments, the MEK inhibitor is trametinib.

In some embodiments, the inhibitor targeting the MAPK pathway is an ERK1/2 inhibitor. In some embodiments, the ERK1/2 inhibitor is SCH772984 or VTX11e.

Also described herein are methods for treating cancer in an individual in need thereof, comprising administration of a JAK inhibitor and an inhibitor targeting the MAPK pathway to the individual and further comprising determining the level of RNF125 in the individual. In some embodiments, the method further comprises determining the level of JAK1 in the individual. In some embodiments, the method further comprises determining the level of EGFR in the individual. In some embodiments, the method further comprises determining the level of AXL in the individual.

Also described herein are methods for treating cancer in an individual in need thereof comprising administration of a JAK inhibitor, a B-Raf inhibitor and an ERK1/2 inhibitor to the individual. Also described herein are methods for treating cancer in an individual in need thereof comprising administration of a JAK inhibitor, a MEK inhibitor and an ERK1/2 inhibitor to the individual. Also described herein are methods for treating cancer in an individual in need thereof comprising administration of a JAK inhibitor, a B-Raf inhibitor, a MEK inhibitor and an ERK1/2 inhibitor to the individual.

Also described herein are methods for treating a BRAF mutant cancer in an individual in need thereof comprising administration of ruxolitinib and dabrafenib to the individual. Also described herein are methods for treating a BRAF mutant cancer in an individual in need thereof comprising administration of ruxolitinib, dabrafenib and trametinib to the individual.

Also described herein are methods for treating cancer in an individual in need thereof comprising determining the level of RNF125 activity or expression in the cancer cells of an individual, and administering to the individual a JAK inhibitor and an inhibitor targeting the MAPK pathway to the individual if the RNF125 activity or expression is reduced in the cancer cells. In some embodiments, the inhibitor targeting the MAPK pathway is a B-Raf inhibitor.

Also described herein are methods of monitoring and treating an individual with a BRAF mutant cancer for resistance to a B-Raf inhibitor comprising determining the level of RNF125 activity or expression in the cancer cells of an individual on a periodic basis, and administering to the individual a JAK inhibitor in combination with an inhibitor targeting the MAPK pathway if the RNF125 activity or expression is reduced in the cancer cells. In some embodiments, the inhibitor targeting the MAPK pathway is a B-Raf inhibitor.

Also described herein are methods of identifying and treating an individual with a BRAF mutant cancer for resistance to a B-Raf inhibitor comprising determining the level of RNF125 activity or expression in the cancer cells of an individual; identifying the individual as being resistant to a B-Raf inhibitor if the RNF125 activity or expression is low in the cancer cells; and administering to the individual a JAK inhibitor and an inhibitor targeting the MAPK pathway to the individual if the RNF125 activity or expression is reduced in the cancer cells. In some embodiments, the inhibitor targeting the MAPK pathway is a B-Raf inhibitor.

Also described herein are methods for treating cancer in an individual having reduced RNF125 activity or expression comprising administration of a JAK inhibitor and an inhibitor targeting the MAPK pathway to the individual. In some embodiments, the inhibitor targeting the MAPK pathway is a B-Raf inhibitor.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates the correlation between RNF125 expression and B-Raf inhibitor resistance in melanoma patients. The expression of RNF125 is shown prior to B-Raf inhibitor treatment (PRE), during B-Raf inhibitor treatment (ON), and after the development of progressive disease (PRO). The number of patients are indicated by n.

FIG. 2 illustrates the RNF125 protein expression levels in tissue samples collected from melanoma patients and stained using anti-RNF125 antibody. The expression of RNF125 is shown prior to B-Raf inhibitor treatment (PRE), and after the development of progressive disease (PRO), visualized under different magnifications (10× and 40×).

FIG. 3 illustrates the correlation between IC50 to B-Raf inhibitors (PLX-4032/PLX-4720) and RNF125 expression. The top panel of the figures shows the growth curves for various UACC cell lines, cultured in the presence of B-Raf inhibitor PLX-4032. The bottom panel of the figure shows the IC50 values, towards the B-Raf inhibitor PLX-4032, for two UACC cells lines with high RNF 125 expression (UACC91 and UACC502), and three UACC cell lines with low RNF125 expression (UACC612, UACC647, and UACC1113).

FIG. 4 illustrates the effect of shRNA mediated knockdown of RNF125 on the growth curves and IC50 values, towards the B-Raf inhibitor, in various UACC melanoma cell lines. (A) The top panels for FIGS. 4A-D show the growth curves for UACC91 cells (A), UACC612 cells (B), UACC647 cells (C), and UACC1113 cells (D). The bottom panel of FIGS. 4A-D show the IC50 values, towards B-Raf inhibitors, for UACC 91 (A), UACC612 (B), UACC647 (C), and UACC1113 (D), cells, expressing either a shRNA specific for RNF125 (shRNF125) or a scrambled shRNA (Scr).

FIG. 5 illustrates the interaction between ectopically expressed FLAG-tagged RNF125 RING mutant (RNF125RM) and V5 tagged-JAK1(JAK1-V5) in HEK293T cells. The top half of the figure shows western blots for cell lysates immunoblotted with anti-RNF125RM or anti-JAK1 antibodies (Lysates, lanes 1-3) and immunoprecipitates that were immunoblotted with anti-FLAG antibody (IP: FLAG, lanes 4-6). The bottom half of the figure shows western blots for cell lysates immunoblotted with anti-RNF125RM or anti-JAK1 antibodies (Lysates, lanes 1-3) and immunoprecipitates that were immunoblotted with anti-V5 antibody (IP:V5, lanes 4-6).

FIG. 6 illustrates the JAK1 levels in A375 cells subjected to cycloheximide chase assay. The top panels of FIGS. 6A and B are western blots, using anti-JAK1 antibodies, for cell lysates, from A375 (A) cells expressing a scrambled shRNA (Scr) or shRNF125 and parenteral or B-Raf inhibitor resistant Lu1205 cells (B), treated with cycloheximide for various lengths of time (0 hr, 1 hr, 2 hr, 4 hr, 8 hr, and 12 hr). β-actin levels are loading control for the western blots.

FIG. 7 illustrates the expression levels of JAK1 and EGFR in UACC1113 cells expressing scrambled shRNA, shRNAs specific towards JAK1 (shJAK1-1, 2, and 3), or shRNA specific towards EGFR (shEGFR). FIG. 7A shows the protein levels determined by immunoblotting of above mentioned shRNA expressing UACC1113 cell lysates with anti-JAK1 or anti-EGFR antibodies. β-actin levels are loading control for the western blots. FIG. 7B shows relative transcript levels for RNF125, JAK1, and EGFR, in UACC1113 cells expressing shRNAs as described above.

FIG. 8 illustrates the essential role of JAK1 and EGFR in maintaining growth in B-Raf inhibitor resistant melanoma cell line UACC91 subjected to doubleknockdowns with shRNAs. FIG. 8A shows the growth curves (top panel) and relative transcript levels (bottom panel) of RNF125 and JAK1 for UACC91 cells expressing the following combinations of shRNAs: two scrambled shRNA (Scr_Scr), a scrambled shRNA and shRNF125 (RNF125_Scr), or shRNF125 and shJAK1 (RNF125_JAK1). FIG. 8B shows the growth curves (top panel) and relative transcript levels (bottom panel) of RNF125 and JAK1 for UACC91 cells expressing the following combinations of shRNAs: two scrambled shRNA (Scr_Scr), a scrambled shRNA and shEGFR (EGFR_Scr), a scrambled shRNA and shJAK1 (JAK1_Scr), or shJAK1 and shEGFR (JAK1 EGFR). The middle panels of FIGS. 8A and B tabulate the IC50 values, towards B-Raf inhibitors, in UACC91 cells expressing various combinations of shRNAs described above.

FIG. 9 illustrates the growth inhibition of B-Raf inhibitor resistant melanoma cells by pharmacological inhibition of JAK1 and EGFR. FIG. 9A shows the growth of A375 cells cultured with (+BRAFi) or without (−BRAFi) the B-Raf inhibitor PLX-4023 alone or in various combinations with EGFR inhibitor gefinitib and JAK inhibitor pyridone 6, as indicated. FIG. 9B shows the growth of Lu1205 cells cultured with (+BRAFi) or without (−BRAFi) the B-Raf inhibitor PLX-4023 alone or in various combinations with EGFR inhibitor gefinitib and JAK inhibitor pyridone 6, as indicated.

### DETAILED DESCRIPTION OF THE INVENTION

Provided herein, in certain embodiments, are methods for treating cancer in a subject with a combination therapy of a JAK inhibitor and an inhibitor targeting the MAPK pathway. Also provided herein are compounds, pharmaceutical compositions and medicaments comprising such compounds for a combination therapy of a cancer.

In certain embodiments, the methods described herein, are for treatment of a cancer resistant to an inhibitor targeting the MAPK pathway in a subject with a combination therapy of a JAK inhibitor and an inhibitor targeting the MAPK pathway. In some instances, the combination therapy is a JAK inhibitor and a B-Raf inhibitor. In certain instances, the combination therapy is a JAK inhibitor and MEK inhibitor. In further instances, the combination therapy is a JAK inhibitor and an ERK1/2 inhibitor. In yet further instances, the combination therapy is a JAK inhibitor, a B-Raf inhibitor and a MEK inhibitor. In other instances, the combination therapy is a JAK inhibitor, a B-Raf inhibitor and an ERK1/2 inhibitor. In yet other instances, the combination therapy is a JAK inhibitor, a B-Raf inhibitor, a MEK inhibitor and an ERK1/2 inhibitor.

In certain instances, the cancer is resistant to B-Raf inhibitors. In further instances, the cancer is resistant to MEK inhibitors. In yet further instances, the cancer is resistant to ERK1/2 inhibitors. In some instances, the cancer is resistant is resistant to more than one inhibitor targeting the MAPK pathway, e.g., B-Raf and MEK inhibitors, B-Raf and ERK1/2 inhibitors, MEK and ERK1/2 inhibitors, and B-Raf, MEK and ERK1/2 inhibitors.

In certain embodiments, the methods described herein, are for treatment of melanoma in a subject with a combination therapy of a JAK inhibitor, and an inhibitor targeting the MAPK pathway (e.g., a B-Raf, MEK, and/or an ERK1/2 inhibitor). In some embodiments, the methods described herein, are for treatment of papillary thyroid cancer, cisplatin-refractory testicular cancer, cholangiocarcinoma, colorectal cancer, ovarian cancer, prostate cancer, glioblastoma, non-small cell lung carcinoma (NSCLC), head and neck squamous cell carcinoma (HNSCC), breast cancer, or pancreatic cancer, with a combination therapy of a JAK inhibitor and an inhibitor targeting the MAPK pathway (e.g., a B-Raf, MEK, and/or a ERK1/2 inhibitor).

### MAPK Signaling in Melanoma Biology

The mitogen-activated protein kinase (MAPK) pathway is important to oncogenic signaling in the patients with metastatic melanoma. Mutations have been identified in neuroblastoma Ras viral (v-ras) oncogene homolog (NRAS), B-Raf protooncogene serine/threonine kinase (BRAF), mitogen-activated protein kinase kinases (MEK1, MEK2), with implications for prognosis and treatment. The development of inhibitors to mediators of the MAPK pathway, including those to CRAF, BRAF, and MEK, led to advances in the treatment of patients with melanoma. In particular, the selective BRAF inhibitor vemurafenib has been shown to improve overall survival in patients with tumors harboring BRAF mutations. However, the duration of benefit is limited in many patients, as they develop resistance to the inhibitors. In the majority of cases, acquired BRAF inhibitor resistance is mediated by reactivation of MAPK signaling via several mechanisms including NRAS (Q61K) mutations, BRAF amplification, activating MEK1 (C121S and P124L) mutations, MEK2 (Q60P) mutations concurrent with BRAF amplification and BRAF splice-form mutants. Simultaneous targeting of BRAF and MEK, which prevents the reactivation of MAPK signaling, has been shown to increase levels of cell death and tumor regression. Clinical trials with the BRAF/MEK inhibitor combination (dabrafenib plus trametinib) has shown increased progression free survival (PFS) compared to BRAF inhibitor alone, as determined by Response Evaluation Criteria in Solid Tumors (RECIST 1.1).

Despite initial promising observation of prolonged PFS, treatment failure still occurs with the development of resistance to the BRAF/MEK inhibitor combination, similar to those seen in patients on BRAF inhibitor monotherapy. Preliminary genetic analysis of selected melanoma patients acquiring resistance to dabrafenib plus trametinib revealed mechanisms of resistance analogous to those seen in patients on BRAF inhibitor monotherapy and highlighted the role of MEK2 Q60P mutations, BRAF-splice mutants and BRAF amplification. It is therefore contemplated that failure on BRAF inhibitor therapy also confers resistance to MEK inhibition, with minimal clinical activity being seen to trametinib in patients failing dabrafenib and a 15% response rate (median PFS 2.8 months) observed in patients failing BRAF inhibitor following treatment with vemurafenib plus cobimetinib, which inhibit B-Raf and MEK respectively.

In light of the recent studies, it is contemplated that further targeted agents in the MAPK signaling pathway can be added to the B-Raf/MEK inhibitor backbone. ERK inhibitors, such as SCH772984, have been shown to overcome acquired resistance to single agent B-Raf and MEK inhibition, in preclinical studies. Another recent finding established the dependency of the MAPK signaling pathway upon copper ions, with the copper-MEK1 interaction being required for efficient ERK phosphorylation. Chelation of copper or the knockdown of the copper transporter CRT1 had anti-proliferative activity against BRAF-mutant melanoma cells and could overcome vemurafenib resistance mediated through the C121S MEK1 mutations.

In some embodiments, the combination therapies described herein are for treatment of subjects with malignant melanoma, using a combination of inhibitors that target one or more genes in the MAPK signaling pathway, e.g. BRAF, MEK1, MEK2, ERK1, ERK2 and the like.

In some embodiments, the combination therapies described herein are for treatment of subjects with a cancer resistant to one or more inhibitors of the MAPK signaling pathway. In certain instances the cancer is resistant to one or more of the following: a B-Raf inhibitor, a MEK inhibitor, or an ERK1 or 2 inhibitor.

### B-Raf

BRAF is a human gene coding for the protein B-Raf. The gene is also referred to as protooncogene B-Raf and v-Raf murine sarcoma viral oncogene homolog B1, while the protein is more formally known as serine/threonine-protein kinase B-Raf. The B-Raf protein is involved in sending signals inside cells, which are involved in directing cell growth. B-Raf inhibition causes programmed cell death in cancer cells (e.g., melanoma cell lines) by inhibition of the B-Raf/MEK/ERK pathway.

Melanoma cases in the US are increasing, with an estimated incidence of >76,000 cases in 2014, with approximately 9,700 deaths. A key discovery is the finding that approximately 50% of melanomas possess an activating mutation in the BRAF gene (primarily T1799A: V600E) (that is, at amino acid position number 600 on the B-Raf protein, the normal valine is replaced by glutamic acid). This discovery, in turn, has allowed for the development of drugs selectively targeting mutant B-Raf. Vemurafenib (also known as Zelboraf®, PLX-4032, RG7-204 or RO-5185426) and dabrafenib (also known as Tafinlar® or GSK2118436) are potent and selective inhibitors of the most common mutant forms of B-Raf. Vemurafenib and dabrafenib have each also received FDA approval for the treatment of late-stage melanoma. Melanoma cells without this mutation are not inhibited by vemurafenib; the drug paradoxically stimulates normal B-Raf and may promote tumor growth in such cases.

In some embodiments, the combination therapies described herein are for treatment of subjects with melanoma harboring the mutation V600E in B-Raf.

Apart from V600E, other cancer associated mutations identified on the B-Raf protein are V600K (valine to lysine at position number 600), K601E (lysine to glutamic acid at position number 601), G469A (glycine to alanine at position number 469), D594G (aspartic acid to glycine at position number 594), V600R (valine to arginine at position number 600), or L597V (leucine to valine at position number 597).

In some embodiments, the combination therapies described herein, are for treatment of subjects harboring one or more of the mutations V600K, K601E, G469A, D594G, V600R, or L597V.

### B-Raf Inhibitors

Vemurafenib has been approved by the FDA for treatment of melanoma. Relapses after treatment with vemurafenib are unfortunately inevitable. Median PFS was approximately 7 months for the initial vemurafenib Phase 1 extension study. The experience with targeted therapies in other diseases such as chronic myelogenous leukemia (CML) and non-small cell lung cancer (NSCLC) suggests that understanding the molecular origins of resistance will lead to improved patient outcomes and the development of effective second-line targeted therapies. Dabrafenib has been approved by FDA for treatment of cancers associated with a mutated version of the gene BRAF.

Dabrafenib acts as an inhibitor of the associated enzyme B-Raf, which plays a role in the regulation of cell growth. Dabrafenib has clinical activity with a manageable safety profile in clinical trials of phase 1 and 2 in patients with BRAF(V600)-mutated metastatic melanoma. Clinical trial data demonstrated that resistance to dabrafenib and other BRAF inhibitors occurs within 6 to 7 months. The BRAF inhibitor dabrafenib has also been approved for combination therapy the MEK inhibitor trametinib.

In some embodiments, a B-Raf inhibitor for combination therapies described herein is dabrafenib.

In additional embodiments a B-Raf inhibitor is selected from

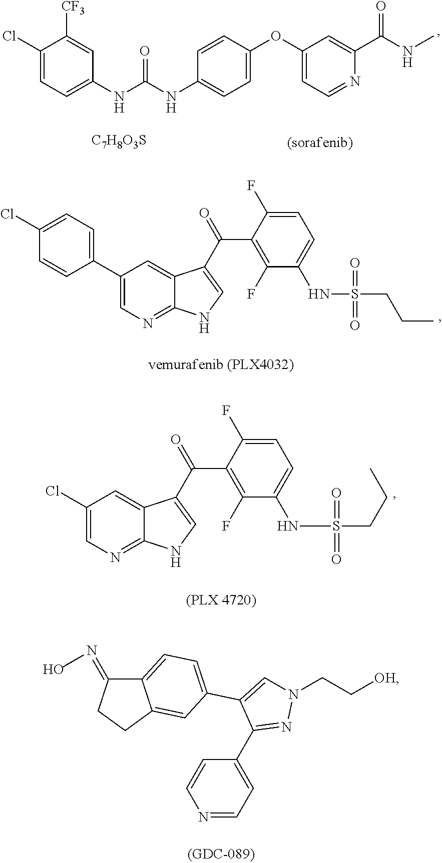

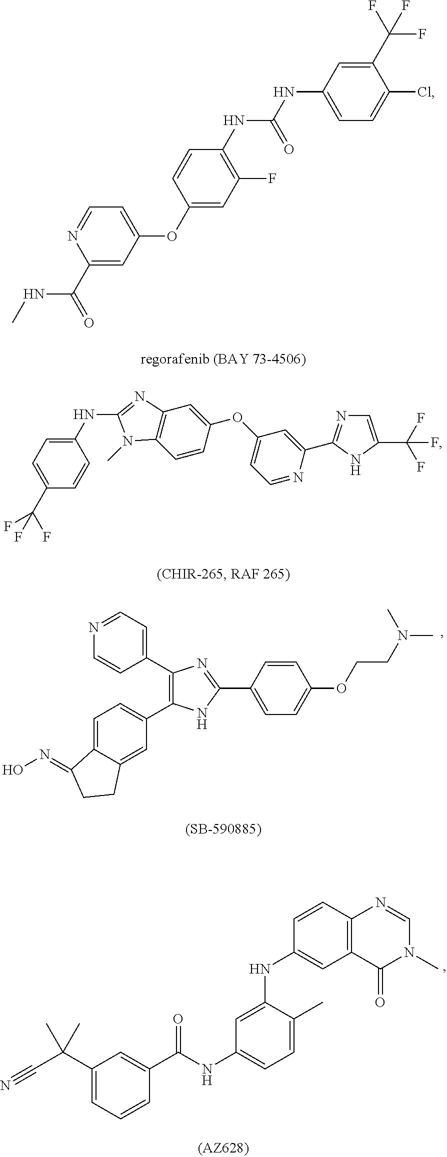 [SMILES: CNC(=O)c1cc(Oc2ccc(NC(=O)Nc3ccc(Cl)c(C(F)(F)F)c3)c(F)c2)ccn1 ; Cn1c(Nc2ccc(C(F)(F)F)cc2)nc2cc(Oc3ccnc(-c4ncc(C(F)(F)F)[nH]4)c3)ccc21 ; CN(C)CCOc1ccc(-c2nc(-c3ccncc3)c(-c3ccc4c(c3)CC/C4=N\O)[nH]2)cc1 ; Cc1ccc(NC(=O)c2cccc(C(C)(C)C#N)c2)cc1Nc1ccc2ncn(C)c(=O)c2c1]

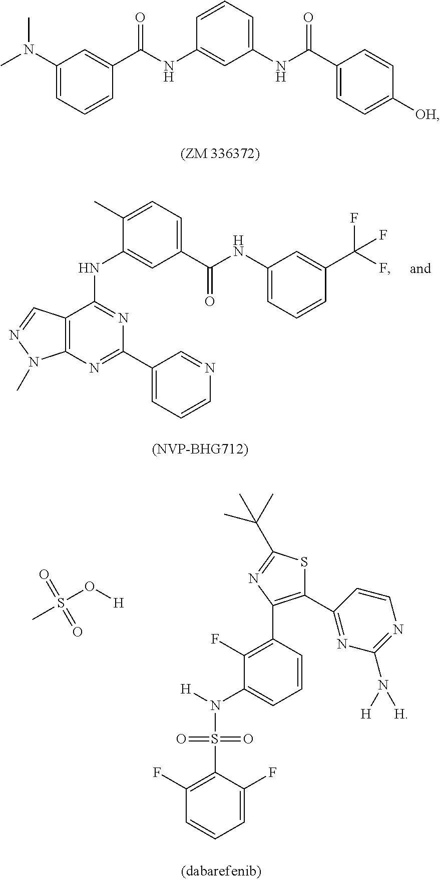

### Mechanisms of Resistance to Selective B-Raf Inhibition

Published clinical data with the first generation selective B-Raf inhibitors have shown antitumor activity in advanced incurable melanoma. Responses have been limited to those patients with V600E B-Raf mutant melanoma. Despite these recent advances, disease progression following treatment with selective B-Raf inhibitors appears inevitable for most patients. Median PFS from published reports is approximately 7 months, although the database is small and therefore the confidence intervals around this point estimate are still broad.

Preclinical insights regarding the mechanisms of resistance to B-Raf inhibitors and analysis of on-treatment tumor biopsies in patients receiving selective BRAF inhibitors suggest that resistance is mechanistically strikingly different from patterns of resistance to other tyrosine kinase inhibitors. In particular, mutations in the target kinase that prevent the drug from binding have not been detected. Instead, a variety of ‘oncogene by-pass’ events emerge that allow the cell to evade the effects of B-Raf pathway blockade by the drug. To date, observations of resistance include the following
BRAF pathway by-pass through activation of RAF1 and COT/TPL2, thereby restoring MEK activation;
Activating mutations in NRAS that restore MEK activation;
Increased activation of the receptor tyrosine kinase PDGFRβ, which acts via an unspecified MEK-independent mechanism;
RAF kinase switching such that cells use the CRAF and ARAF isoforms to activate the MAPK pathway;
Enhanced IGF-1R and PI-3K/AKT activity in melanoma cell lines resistant to BRAF inhibitors. Preclinical studies combining IGF-1R/PI-3K and MEK inhibitors induced death of BRAF inhibitor-resistant cells;
In a tumor sample obtained at the time of progression, homozygous deletion of PTEN was observed, a mutation that was not present in the pre-treatment biopsy;
MEK1 mutation also has been reported as a mechanism of resistance;
Novel BRAF(V600E) splicing variants lacking the RAS-binding domain resulting in a novel form of BRAF(V600E) that facilitates enhanced BRAF dimerization with low RAS activation and ERK signaling in the presence of vemurafenib.

A variety of clinical study strategies are contemplated to prevent and/or overcome resistance to B-Raf inhibitors, including combining selective B-Raf and MEK inhibitors or studies designed to overcome the induced oncogene by-pass events. However, Applicants have undertaken a different and unique approach. However, combination therapy using targeted agents presents numerous challenges.
1. In particular, it is ultimately likely that combination therapy targeting multiple pathways will be required in order to optimize patient therapy and prolong survival
2. Combining targeted agents is not always straightforward. The drugs do have significant toxicities alone and with sometimes unpredictable and severe side effects in combination. Careful Phase 1 studies with a limited number of experimental agents being given at the same time with PK and pharmacodynamic endpoints are mandatory before more elaborate regimens can be contemplated and evaluated in the clinic
3. Strategies to prospectively identify patients that will benefit from novel combinations as a first line strategy, or at the time of progression on vemurafenib therapy, are currently unavailable.

Given the unpredictable nature of combination therapy, and unpredictable reasons for development of resistance to B-Raf inhibitors in the clinic, Applicants explored combination therapy for treatment of cancer using dabrafenib and trametenib in combination with a JAK inhibitor (e.g., ruxolitinib).

### MEK

Classic activation of the Ras/Raf/MEK/MAPK cascade occurs following ligand binding to a receptor tyrosine kinase at the cell surface, but a vast array of other receptors have the ability to activate the cascade as well, such as integrins, serpentine receptors, heterotrimeric G-proteins, and cytokine receptors. Although conceptually linear, considerable cross talk occurs between the Ras/Raf/MEK/MAPK pathway and other MAPK pathways as well as many other signaling cascades. The pivotal role of the Ras/Raf/MEK/MAPK pathway in multiple cellular functions underlies the importance of the cascade in oncogenesis and growth of transformed cells. Ras activation is the first step in activation of the cascade. Following Ras activation, Raf (A-Raf, B-Raf, or Raf-1) is recruited to the cell membrane through binding to Ras and activated in a complex process involving phosphorylation and multiple cofactors. Raf proteins directly activate MEK1 and MEK2 via phosphorylation of multiple serine residues. MEK1 and MEK2 are themselves tyrosine and threonine/serine dual-specificity kinases that subsequently phosphorylate threonine and tyrosine residues in Erk1 and Erk2 resulting in activation.

MEK inhibitor are chemicals or drugs that inhibit the mitogen-activated protein kinase kinase enzymes MEK1 and/or MEK2 and are useful in cancer treatment. They can be used to affect the MAPK/ERK pathway which is often overactive in some cancers. Hence, MEK inhibitors have potential for treatment of some cancers, especially BRAF-mutated melanoma, and KRAS/BRAF mutated colorectal cancer. Examples of MEK inhibitors include: Trametinib (GSK1120212), for treatment of BRAF-mutated melanoma and possible combination with BRAF inhibitor dabrafenib to treat BRAF-mutated melanoma; Selumetinib, for non-small cell lung cancer (NSCLC); Binimetinib (MEK162), for biliary tract cancer and melanoma; PD-325901, for breast cancer, colon cancer, and melanoma; Cobimetinib (XL518); CI-1040 and PD035901.

Trametinib is a MEK inhibitor drug approved by FDA with inhibitory activity towards MEK1 and MEK2. Trametinib, alone as single-agent or in combination with B-Raf inhibitor dabrafenib has been approved by FDA for treatment of metastatic melanoma carrying the B-Raf V600E mutation. Clinical trial data demonstrated that progression on single-agent trametinib occurs within 6 to 7 months.

### ERK

ERK1/2 are kinases that act directly downstream of MEK. ERK1 and ERK2 kinases are about 85% identical in their amino-acid sequence and are activated by phosphorylation on tyrosine and threonine residues catalyzed by MEK. Activated ERK regulates a number of cellular events, including cell proliferation and survival. Resistance to RAF and MEK inhibitors has been shown to involve the recovery of ERK signaling, which suggests that use of an ERK inhibitor can help the potency and durability of ERK pathway inhibition.

SCH772984 is an ATP-competitive ERK1 and ERK2 inhibitor that was derived from an affinity-based high-throughput screen for small molecules that bind to the unphosphorylated (inactive) form of ERK2. It has been shown to inhibit purified ERK1 and ERK2 with IC50 values of 4 nM and 2 nM, respectively, and also displays a marked selectivity for inhibiting ERK1 and ERK2 in biochemical and cellular assays. It is hypothesized that, binding of SCH772984 to ERK results in a dual mechanism of inhibition: inhibition of ERK1 and ERK2 intrinsic kinase activity and the prevention of phosphorylation of ERK1 and ERK2 by MEK. Studies conducted with SCH772984 indicate that the small molecule inhibits cellular proliferation and causes apoptosis selectively in tumour cell lines that carry RAS or BRAF mutations, and also induces significant tumour regressions in mice with BRAF- or RAS-mutant xenografts. SCH772984 also demonstrated inhibition activity in cells that were resistant to either BRAF or MEK inhibitors and in cells that became resistant to the dual combination of these inhibitors.

MK-8353/SCH900353, a clinical grade analogue of SCH772984, is currently being tested in Phase I clinical trials. Two other ERK inhibitors, BVD-523 (Biomed Valley Discoveries) and RG7842 (GDC0994; Genentech/Roche), have recently entered clinical trials, but their properties and preclinical activities are not available in the public domain.

### JAK/JAK1

The Janus kinase (JAK) family is a group of tyrosine kinases, and includes JAK1, JAK2, JAK3, and TYK2. The JAK family transmits cytokine-mediated signals into cells by JAK-STAT (Signal Transducer and Activator of Transcription) pathways. JAK1 is a key regulator of immune cell activation and interferon responses. However, there is limited information regarding JAK1 regulation by the ubiquitin proteasome system (UPS) or the importance of this activity in melanoma cell chemoresistance.

In some embodiments, JAK1 is regulated by the ubiquitin ligase RNF125. In some embodiments, RNF125 expression decreases JAK1 protein steady state levels. In some embodiments, RNF125 E3 ligase is required for RNF125 expression to decrease JAK1 protein steady state levels. In some embodiments, transcriptional silencing of RNF125 in resistant melanomas accounts for the upregulation of JAK1.

In some embodiments, JAK1 is the component responsible for the upregulation of RTK. In some embodiments, treatment of B-Raf inhibitor resistant melanomas with JAK inhibitors effectively inhibits their ability to proliferate, such as to form colonies and grow on soft agar. In some embodiments, the JAK inhibitors are pyridone 6 and AZD1480. In some embodiments, PLX4032 (vemurafenib) in combination with pyridone 6 and AZD1480 elicits an even greater response.

JAK inhibitors inhibit the activity of one or more of the JAK family of enzymes (JAK1, JAK2, JAK3, TYK2), thereby interfering with the JAK-STAT signaling pathway. These inhibitors have therapeutic application in the treatment of cancer and inflammatory diseases. Cytokines play key roles in controlling cell growth and the immune response. Many cytokines function by binding to and activating type I and type II cytokine receptors. These receptors in turn rely on the Janus kinase (JAK) family of enzymes for signal transduction. Therefore, drugs that inhibit the activity of Janus kinases block cytokine signaling. More specifically, Janus kinases phosphorylate activated cytokine receptors. These phosphorylated receptors in turn recruit STAT transcription factors which modulate gene transcription.

The first JAK inhibitor to reach clinical trials was tofacitinib. Tofacitinib is a specific inhibitor of JAK3 (IC50=2 nM) thereby blocking the activity of IL-2, IL-4, IL-15 and IL-21. Hence, Th2 cell differentiation is blocked and therefore tofacitinib is effective in treating allergic diseases. Tofacitinib to a lesser extent also inhibits JAK1 (IC50=100 nM) and JAK2 (IC50=20 nM) which in turn blocks IFN-γ and IL-6 signaling and consequently Th1 cell differentiation. Examples of JAK inhibitors include: Ruxolitinib against JAK1/JAK2 for psoriasis, myelofibrosis, and rheumatoid arthritis; Tofacitinib (tasocitinib; CP-690550) against JAK3 for psoriasis and rheumatoid arthritis; Baricitinib (LY3009104, INCB28050) against JAK1/JAK2 for rheumatoid arthritis; CYT387 against JAK2 for myeloproliferative disorders; Lestaurtinib against JAK2, for acute myelogenous leukemia (AML); Pacritinib (SB1518) against JAK2 for relapsed lymphoma and advanced myeloid malignancies, chronic idiopathic myelofibrosis (CIMF); and TG101348 against JAK2 for myelofibrosis.

Pyridone 6 is a cell-permeable JAK inhibitor. Pyridone 6 is a potent, reversible, ATP-competitive JAK inhibitor. It inhibits JAK1, 2, and 3 with IC₅₀ values of 15, 1, and 5 nM, respectively. It displays significantly weaker affinities (IC₅₀s=130 nM->10 μM) for other protein tyrosine kinases. It was shown to block IL-2 and IL-4-dependent proliferation of mouse T-cell lymphoma cells with IC₅₀ values of 50-100 nM. AZD1480 is a novel ATP-competitive inhibitor of JAK1 and JAK2 with IC₅₀ of 1.3 nM and 0.26 nM, respectively. PLX4032 (vemurafenib) is a cell-permeable JAK inhibitor. PLX4032 is a potent, selective and ATP-competitive, inhibitor of BRAF(V600E) kinase with potential antineoplastic activity. PLX4032 selectively binds to the ATP-binding site of BRAF(V600E) kinase and inhibits its activity, which may result in an inhibition of an over-activated MAPK signaling pathway downstream in BRAF(V600E) kinase-expressing tumor cells and a reduction in tumor cell proliferation.

In some embodiments, inhibition of JAK in B-Raf inhibitor (BRAFi) resistant tumors sensitizes them to cell death programs and overcomes the resistant phenotype. In some embodiments, these tumors are melanoma. In some embodiments, described herein are methods for inhibiting proliferation of tumors that are resistant to B-Raf inhibitor.

### Inhibition of JAK in B-Raf Inhibitor or B-Raf and MEK Inhibitor Resistant Cancers

In some embodiments, inhibition of JAK in MEK inhibitor (MEKi) resistant tumors sensitizes them to cell death programs and overcomes the resistant phenotype. In some embodiments, these tumors are melanoma. In some embodiments, described herein are methods for inhibiting proliferation of tumors that are resistant to MEKi.

In some embodiments, inhibition of JAK in B-Raf and MEK inhibitor resistant tumors sensitizes them to cell death programs and overcomes the resistant phenotype. In some embodiments, these tumors are melanoma. In some embodiments, described herein are methods for inhibiting proliferation of tumors that are resistant to B-Raf and MEK inhibitor.

In some embodiments, described herein are methods for inhibiting proliferation of tumors that are resistant to B-Raf and MEK inhibitors, by inhibition of JAK, and one or more components of MAPK signaling pathway.

In some embodiments, described herein are methods for inhibiting proliferation of tumors that are resistant to B-Raf and MEK inhibitors, by inhibition of JAK, and one or more components of MAPK signaling pathway, selected from a group consisting of BRAF, MEK, and ERK1/2.

### Diagnosis of Cancer

In some embodiments, described herein are methods of diagnosis of cancer. A diagnosis or diagnostic, as used herein, is a compound, method, system, or device that assists in the identification and characterization of a health or disease state. The diagnosis can be performed using assays as known in the art.

In some embodiments, RNF125 is used as a biomarker in the diagnosis of cancer. As used herein, the terms “biomarker” or “diagnosis marker” is intended to indicate a substance capable of diagnosing cancer by distinguishing cancer cells or subject suffering from cancer from normal cells or subjects, and includes organic biological molecules, quantities of which are increased or decreased in cancer cells relative to normal cells, such as polypeptides, proteins or nucleic acids (e.g., mRNA, etc.), lipids, glycolipids, glycoproteins and sugars (monosaccharides, disaccharides, oligosaccharides, etc.). As described herein, levels of RNF125 polypeptide or a polynucleotide coding therefore, which are specifically expressed at low levels in cancer cells, relative to normal cells or tissues, may indicate resistance to BRAF inhibitor therapy, or MEK inhibitor therapy, or a combination thereof, in tumor specimens.

In some embodiments, a method for treating cancer is described wherein the level of RNF125 activity or expression in the cancer cells of an individual is determined, and then a JAK inhibitor, or an inhibitor targeting the MAPK pathway, or a combination of a JAK inhibitor and an inhibitor targeting the MAPK pathway is administered to the individual if the level of RNF125 is reduced in the cancer cells. In some embodiments, the level of RNF125 is compared to a control sample. In some embodiments, the inhibitors targeting the MAPK pathway is selected from a group consisting of a B-Raf inhibitor, a MEK inhibitor, or an ERK1/2 inhibitor. In some embodiments, the inhibitor targeting the MAPK pathway is a B-Raf inhibitor.

In some embodiments, a method for treating cancer is described wherein the level of RNF125 activity or expression in the cancer cells of an individual is determined, and then a JAK inhibitor, or an inhibitor targeting the MAPK pathway, or a combination of a JAK inhibitor and one or more inhibitors targeting the MAPK pathway is administered to the individual if the level of RNF125 is reduced in the cancer cells. In some embodiments, the level of RNF125 is compared to a control sample. In some embodiments, the one or more inhibitors targeting the MAPK pathway is selected from a group consisting of a B-Raf inhibitor, a MEK inhibitor, or an ERK1/2 inhibitor.

The step of determining the level of activity or expression of RNF125 may include measuring the level of RNF125 mRNA transcripts expressed by cells in the sample. This can be done with any suitable method, including but not limited to polymerase chain reaction (PCR), reverse transcriptase-PCR (RT-PCR), real-time polymerase chain reaction (or quantitative real-time polymerase chain reaction), in situ hybridization, Northern blot, sequence analysis, gene microarray analysis and/or the detection of a reporter gene. In some embodiments, the level of RNF125 protein expression is measured. The level of RNF125 protein expression can be measured with any suitable method, including but not limited to immunoblot, enzyme-linked immunosorbant assay (ELISA), radioimmunoassay (RIA), immunoprecipitation, immunohistochemistry and immunofluorescence. In some embodiments, the level of RNF125 biological activity in the sample is measured. The level of RNF125 biological activity can be measured with any suitable method, including but not limited to measuring proliferation of cells expressing RNF125, detecting DNA synthesis in cells expressing RNF125, measuring ubiquitination of JAK1 in cells expressing RNF125, and measuring EGFR expression in cells expressing RNF125.

### Treatment of Cancer

Cancers treatable by combination therapies described herein include, but are not limited to, breast cancer, lung cancer, head and neck cancer, brain cancer, abdominal cancer, colon cancer, colorectal cancer, esophageal cancer, parapharyngeal cancer, gastrointestinal cancer, glioma, liver cancer, tongue cancer, neuroblastoma, osteosarcoma, ovarian cancer, renal cancer, pancreatic cancer, retinoblastoma, cervical cancer, uterine cancer, Wilm's tumor, multiple myeloma, skin cancer, lymphoma, leukemia, blood cancer, anaplastic thyroid tumor, sarcoma of the skin, melanoma, adenocystic tumor, hepatoid tumor, non-small cell lung cancer, chondrosarcoma, pancreatic islet cell tumor, prostate cancer including castration resistant forms, ovarian cancer, and/or carcinomas including but not limited to squamous cell carcinoma of the head and neck, colorectal carcinoma, glioblastoma, cervical carcinoma, endometrial carcinoma, gastric carcinoma, pancreatic carcinoma, leiomyosarcoma and breast carcinoma. In some embodiments, the combination therapies described herein treat a lung cancer such as non-small cell lung cancer (NSCLC). In other embodiments, the combination therapies described herein treat a head and neck cancer such as head and neck squamous cell (HNSCC). In other embodiments, the combination therapies described herein treat a melanoma. In other embodiments, the combination therapies described herein treat a B-Raf inhibitor resistant melanoma. In other embodiments, the combination therapies described herein treat a B-Raf inhibitor-naïve melanoma.

The combination therapies described herein treat various stages of cancer including stages which are locally advanced, metastatic and/or recurrent. In cancer staging, locally advanced is generally defined as cancer that has spread from a localized area to nearby tissues and/or lymph nodes. In the Roman numeral staging system, locally advanced usually is classified in Stage II or III. Cancer which is metastatic is a stage where the cancer spreads throughout the body to distant tissues and organs (stage IV). Cancer designated as recurrent generally is defined as the cancer has recurred, usually after a period of time, after being in remission or after a tumor has visibly been eliminated. Recurrence can either be local, i.e., appearing in the same location as the original, or distant, i.e., appearing in a different part of the body. In certain instances, a cancer treatable by combination therapies described herein is unresectable, or unable to be removed by surgery. In further instances, a cancer treatable by the combination therapies described herein is incurable, i.e., not treatable by current treatment methods.

In some embodiments, the combination therapies described herein are administered as a first-line or primary therapy, i.e. subjects are treatment naïve. Other subjects suitable for treatment by the combination therapies described herein include those that have completed first-line anti-cancer therapy. First-line anti-cancer therapies include chemotherapy, radiotherapy, immunotherapy, gene therapy, hormone therapy, surgery or other therapies that are capable of negatively affecting cancer in a patient, such as for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

In additional embodiments, subjects suitable for treatment by the combination therapies described herein include those that are administered a combination of a JAK inhibitor and an inhibitor targeting the MAPK pathway, in combination with one or more than one additional therapy selected from chemotherapy, radiotherapy, immunotherapy, gene therapy, hormone therapy, surgery and/or other therapies that are capable of negatively affecting cancer in a patient, such as for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. In some embodiments, the inhibitor targeting the MAPK pathway is a B-Raf inhibitor.

Chemotherapies for first-line and subsequent therapy include, but are not limited to, hormone modulators, androgen receptor binding agents (e.g., anti-androgens, bicalutamide, flutamide, nilutamide, MDV3100), gonadotropin-releasing hormone agonists and antagonists (e.g., leuprolide, buserelin, histrelin, goserelin, deslorelin, nafarelin, abarelix, cetrorelix, ganirelix degarelix), androgen synthesis inhibitors (abiraterone, TOK-001), temozolomide, mitozolomide, dacarbazine, cisplatin (CDDP), carboplatin, procarbazine, mechlorethamine, cyclophosphamide, camptothecin, ifosfamide, melphalan, chlorambucil, busulfan, nitrosurea, dactinomycin, anthracyclines (e.g., daunorubicin, doxorubicin, epirubicin, idarubicin), bleomycin, plicomycin, mitomycin, etoposide (VP16), tamoxifen, raloxifene, estrogen receptor binding agents, cabazitaxel, paclitaxel, gemcitabine, navelbine, farnesyl-protein transferase inhibitors, transplatinum, 5-fluorouracil, capecitabine, vincristin, vinblastin and methotrexate, topoisomerase inhibitors (e.g., irinotecan, topotecan, camptothecin, etoposide) or any derivative related agent of the foregoing. Many of the above agents are also referred to as hormone therapy agents such as, for example, androgen receptor binding agents, gonadotropin-releasing hormone agonists and antagonists, androgen synthesis inhibitors, estrogen receptor binding agents as well as aromatase inhibitors.

Radiotherapies for first-line and subsequent therapy include factors that cause DNA damage and include what are commonly known as γ-rays, X-rays, and/or the directed delivery of radioisotopes to tumor cells. Other forms of DNA damaging factors include microwaves and UV-irradiation. It is likely that all of these factors affect a broad range of damage on DNA, on the precursors of DNA, on the replication and repair of DNA, and on the assembly and maintenance of chromosomes. Dosage ranges for X-rays may range from daily doses of 50 to 200 roentgens for prolonged periods of time (e.g., 3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Immunotherapies generally rely on the use of immune effector cells and molecules to target and destroy cancer cells. The immune effector may be, for example, a tumor antigen or an antibody specific for some marker on the surface of a tumor cell. The tumor antigen or antibody alone may serve as an effector of therapy or it may recruit other cells to actually effect cell killing. An antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. Alternatively, an tumor antigen may stimulate a subject's immune system to target the specific tumor cells using cytotoxic T cells and NK cells. Immunotherapies include Sipuleucel-T (Provenge®), bevacizumab and the like.

A gene therapy includes a therapeutic polynucleotide is administered before, after, or at the same time as a combination therapy. Therapeutic genes may include an antisense version of an inducer of cellular proliferation (oncogene), an inhibitor of cellular proliferation (tumor suppressor), or an inducer of programmed cell death (pro-apoptotic gene).

Surgery of some type is performed for resectable cancers. Surgery types include preventative, diagnostic or staging, curative and palliative surgery and can be performed as a first-line and subsequent therapy.

In some embodiments, the combination therapies described herein are administered as a second-line therapy after a first-line therapy becomes ineffective or the cancer is recurrent. In other embodiments, the combination therapies described herein administered as a third-line therapy after the first- and second-line therapy fails. In further embodiments, individuals are preselected for having completed a first- or second-line therapy. In some instances, the combination therapies described herein are administered to patients for whom prior platinum-based therapy has failed. In other instances, the combination therapies described herein are administered to patients for whom prior irinotecan therapy has failed.

Subjects, in some embodiments, can also be prescreened or preselected for sensitivity and/or effectiveness of the combination therapies described herein. A subject can be examined for certain biomarkers that allow the subject to be amenable to a combination therapy. Exemplary biomarkers include, but are not limited to mutation and expression status of E3 ubiquitin protein ligase (RNF125), epidermal growth factor receptor (EGFR), Fos-like antigen 2 (FOSL2), platelet derived growth factor C (PDGFC), vascular endothelial growth factor C (VEGFC), collagen type 5 alpha 1 (COL5A1), B-cell lymphoma 2 like 1 (BCL2L1), interferon regulator factor 1 (IRF1), serpine peptidase inhibitor clade E (SERPINE1), Axl receptor tyrosine kinase (AXL), neuropilin 1 (NRP1), fibronectin 1 (FN1), sex determining region Y box 10 (SOX10).

### Patient Cohorts for Combination Therapy

In some embodiments, the subjects treated with combination therapies described herein are cancer patients. In some embodiments, the cancer patients have previously been treated with a B-Raf inhibitor alone or in combination with a MEK inhibitor, and are B-Raf inhibitor or B-Raf and MEK inhibitor resistant. In some embodiments, the cancer patients have not been previously treated with a B-Raf inhibitor, and are B-Raf-naïve. In some embodiments, the cancer patients have not been previously treated with a B-Raf inhibitor, alone or in combination with a MEK inhibitor, and are B-Raf-naïve.

### Pharmaceutical Compositions for Combination Therapy

Disclosed herein, in certain embodiments, are pharmaceutical compositions and formulations comprising: (a) one or more inhibitor(s) targeting the MAPK pathway; (b) a JAK/JAK1 inhibitor; and (c) a pharmaceutically-acceptable excipient. In some embodiments the one or more inhibitor(s) targeting the MAPK pathway is selected from a group consisting of a B-Raf inhibitor, a MEK inhibitor, and an ERK1/2 inhibitor.

Disclosed herein, in certain embodiments, are pharmaceutical compositions and formulations comprising: (a) an inhibitor targeting the MAPK pathway; (b) a JAK/JAK1 inhibitor; and (c) a pharmaceutically-acceptable excipient. In some embodiments the inhibitor targeting the MAPK pathway is a B-Raf inhibitor. In some embodiments the inhibitor targeting the MAPK pathway is a MEK inhibitor. In some embodiments the inhibitor targeting the MAPK pathway is an ERK1/2 inhibitor.

Disclosed herein, in certain embodiments, are pharmaceutical compositions and formulations comprising: (a) a B-Raf inhibitor; (b) a MEK inhibitor; (c) a JAK/JAK1 inhibitor; and (c) a pharmaceutically-acceptable excipient. In some embodiment, the B-Raf inhibitor is dabrafenib. In some embodiments, the JAK/JAK1 inhibitor is ruxolitinib. In some embodiments, the MEK inhibitor is trametinib. In some embodiments, the combination of a B-Raf inhibitor, a MEK inhibitor, and a JAK/JAK1 inhibitor exert a synergistic effect. In some embodiments, the combination of dabrafenib, trametinib, and ruxolitinib exert a synergistic effect. In some embodiments, the combination of dabrafenib, trametinib, and ruxolitinib exert an additive effect. In some embodiments, the combination of dabrafenib, trametinib, and ruxolitinib exert an antagonistic effect. In some embodiments, a combination index (CI) value is used to indicate the behavior of the combination of dabrafenib, trametinib, and ruxolitinib.

### Methods of Administration

In some embodiments of the methods provided herein, a JAK inhibitor and an inhibitor targeting the MAPK pathway are administered simultaneously. In other embodiments, a JAK inhibitor and an inhibitor targeting the MAPK pathway are administered sequentially. In further embodiments, a JAK inhibitor and an inhibitor targeting the MAPK pathway are administered in a single composition. In some embodiments, the inhibitor targeting the MAPK pathway is a B-Raf inhibitor.

In some embodiments of the methods provided herein, administration of a JAK inhibitor and an inhibitor targeting the MAPK pathway is by injection, transdermal, nasal, pulmonary, vaginal, rectal, buccal, ocular, otic, local, topical, or oral delivery. In certain instances, injection is intramuscular, intravenous, subcutaneous, intranodal, intratumoral, intracisternal, intraperitoneal, or intradermal. In some embodiments, the inhibitor targeting the MAPK pathway is a B-Raf inhibitor.

In some embodiments of the methods provided herein, a JAK inhibitor and an inhibitor targeting the MAPK pathway are administered via different routes of administration. In certain embodiments, a JAK inhibitor and an inhibitor targeting the MAPK pathway are administered orally. In some embodiments, a JAK inhibitor and an inhibitor targeting the MAPK pathway are administered in a capsule form. In some embodiments, a JAK inhibitor and an inhibitor targeting the MAPK pathway are administered in about 0.1 to about 12 mg. In some instances, a JAK inhibitor and an inhibitor targeting the MAPK pathway are administered daily. In some embodiments, the inhibitor targeting the MAPK pathway is a B-Raf inhibitor.

In some embodiments of the methods provided herein, a JAK inhibitor and an inhibitor targeting the MAPK pathway are administered orally. In some embodiments, a JAK inhibitor and an inhibitor targeting the MAPK pathway are administered periodically every three weeks. In some embodiments, a JAK inhibitor and an inhibitor targeting the MAPK pathway are administered weekly. In some embodiments, a JAK inhibitor and an inhibitor targeting the MAPK pathway are administered daily. In some embodiments, the inhibitor targeting the MAPK pathway is a B-Raf inhibitor.

In some embodiments, the administration is over a period of time selected from the group consisting of at least about 3 weeks, at least about 6 weeks, at least about 9 weeks, at least about 12 weeks, at least about 15 weeks, at least about 18 weeks, at least about 21 weeks, at least about 24 weeks, at least about 27 weeks, at least about 30 weeks, at least about 33 weeks, at least about 36 weeks, at least about 39 weeks, at least about 42 weeks, at least about 45 weeks, at least about 48 weeks, at least about 51 weeks, at least about 54 weeks, at least about 57 weeks, at least about 60 weeks, at least about 75 weeks, at least about 90 weeks, and at least about 120 weeks.

In some embodiments of the methods provided herein, a JAK inhibitor and an inhibitor targeting the MAPK pathway are provided in a kit. In some embodiments, a kit may comprise a JAK inhibitor and an inhibitor targeting the MAPK pathway and a container (e.g., a vial, ampule, bottle, syringe, and/or dispenser package, or other suitable container). In some embodiments, provided kits may optionally further include a second container comprising a pharmaceutical excipient for dilution or suspension of a provided pharmaceutical composition or compound, such as a JAK inhibitor and an inhibitor targeting the MAPK pathway. In some embodiments, a provided pharmaceutical composition or compound, such as a JAK inhibitor and an inhibitor targeting the MAPK pathway are provided in the container and the second container are combined to form one unit dosage form. In some embodiments, a provided kit further includes instructions for use. In some embodiments, the inhibitor targeting the MAPK pathway is a B-Raf inhibitor.

For oral administration, a JAK inhibitor and an inhibitor targeting the MAPK pathway can be formulated readily by combining the active compounds with pharmaceutically acceptable carriers or excipients well known in the art. Such carriers enable the compounds described herein to be formulated as tablets, powders, pills, dragees, capsules, liquids, gels, syrups, elixirs, slurries, suspensions and the like, for oral ingestion by a patient to be treated. In some embodiments, the inhibitor targeting the MAPK pathway is a B-Raf inhibitor

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. In some embodiments, a JAK inhibitor and an inhibitor targeting the MAPK pathway are in powder form and is directly filled into hard gelatin capsules.

For buccal or sublingual administration, the compositions may take the form of tablets, lozenges, or gels formulated in conventional manner.

Injectable compositions may involve for bolus injection or continuous infusion. An injectable composition of a JAK inhibitor and an inhibitor targeting the MAPK pathway. In some embodiments, the inhibitor targeting the MAPK pathway may be in a form suitable for parenteral or any other type of injection as a sterile suspensions, solutions or emulsions in oily or aqueous vehicles, and may contain formulatory agents such as suspending, stabilizing and/or dispersing agents. The composition may be formulated for intramuscular, intravenous, subcutaneous, intranodal, intratumoral, intracisternal, intraperitoneal, and/or intradermal injection. Pharmaceutical formulations for injection administration include aqueous solutions of the active compounds in water soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

In various embodiments, a JAK inhibitor and an inhibitor targeting the MAPK pathway composition is in liquid form for ocular or otic delivery. Liquid forms include, by way of non-limiting example, neat liquids, solutions, suspensions, dispersions, colloids, foams and the like and can be formulated by known methods.

A JAK inhibitor and an inhibitor targeting the MAPK pathway can be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

Formulations suitable for transdermal administration of a JAK inhibitor and an inhibitor targeting the MAPK pathway may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the JAK inhibitor and the inhibitor targeting the MAPK pathway can be accomplished by means of iontophoretic patches and the like. Additionally, transdermal patches can provide controlled delivery of the JAK inhibitor and the inhibitor targeting the MAPK pathway. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

For administration by inhalation for pulmonary or nasal delivery, a JAK inhibitor and an inhibitor targeting the MAPK pathway may be in the form of an aerosol, a mist or a powder. Pharmaceutical compositions of a JAK inhibitor and an inhibitor targeting the MAPK pathway are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, such as, by way of example only, gelatin for use in an inhaler or insufflator may be formulated containing a powder mix of the compound and a suitable powder base such as lactose or starch.

A JAK inhibitor and an inhibitor targeting the MAPK pathway may also be formulated in rectal or vaginal compositions such as enemas, douches, gels, foams, aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

One may administer a JAK inhibitor and an inhibitor targeting the MAPK pathway in a local rather than systemic manner, for example, via injection of the compound directly into an organ, often in a depot or sustained release formulation. Furthermore, one may administer pharmaceutical composition containing a JAK inhibitor and an inhibitor targeting the MAPK pathway in a targeted drug delivery system, for example, in a liposome coated with organ-specific antibody. The liposomes will be targeted to and taken up selectively by the organ. Pharmaceutical compositions of a JAK inhibitor and an inhibitor targeting the MAPK pathway may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. Pharmaceutical compositions comprising a JAK inhibitor and an inhibitor targeting the MAPK pathway inhibitor may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one pharmaceutically acceptable carrier, diluent or excipient and a JAK inhibitor and an inhibitor targeting the MAPK pathway described herein as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, a JAK inhibitor and a B-Raf inhibitor may exist as tautomers. All tautomers are included within the scope of the compounds presented herein. Additionally, a JAK inhibitor and an inhibitor targeting the MAPK pathway described herein can exist in unsolvated as well as solvated forms with pharmaceutically acceptable solvents such as water, ethanol, and the like. The solvated forms of a JAK inhibitor and an inhibitor targeting the MAPK pathway presented herein are also considered to be disclosed herein. In addition, the pharmaceutical compositions may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions can also contain other therapeutically valuable substances.

Methods for the preparation of compositions comprising a JAK inhibitor and an inhibitor targeting the MAPK pathway described herein include formulating the JAK inhibitor and the inhibitor targeting the MAPK pathway with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The compositions may be in liquid solutions or suspensions, solid forms suitable for solution or suspension in a liquid prior to use, or as emulsions. These compositions may also contain minor amounts of nontoxic, auxiliary substances, such as wetting or emulsifying agents, pH buffering agents, and so forth.

Further forms of pharmaceutical compositions of a JAK inhibitor and an inhibitor targeting the MAPK pathway can be integrated with other active agents in a unitary dosage form for combination therapies. The unitary dosage forms can be formulated to release where both agents are released simultaneously or where there is sequential release of each agent via known modified release mechanisms including but not limited to timed release, delayed release, pH release, pulsatile release and the like.

In further embodiments of the methods provided herein, the subject is preselected for having completed first-line anti-cancer therapy. In other embodiments, subject is preselected for sensitivity to administration of the compound. In certain instances, preselection is by assessment of genetic mutations in JAK or MAPK pathway genes. In certain instances, preselection is by assessment of genetic mutations in JAK or BRAF genes. In other embodiments of the methods provided herein, the methods further comprise evaluating the treated subject, wherein the evaluation comprises determining at least one of: (a) tumor size, (b) tumor location, (c) nodal stage, (d) growth rate of the cancer, (e) survival rate of the subject, (0 changes in the subject's cancer symptoms, (g) changes in the subject's 5-100B concentration, (h) changes in the subject's 5-100B concentration doubling rate, (i) changes in the subject's biomarkers, or (j) changes in the subject's quality of life.

### EXAMPLES

### Example 1: Downregulation of Ubiquitin Ligase RNF125 Confers Resistance of Melanoma Cells to B-Raf Inhibitors Via JAK1 and EGFR

Materials and Methods:

Development of Melanoma Cells Resistant to B-Raf Inhibitors

Melanoma cells A375 and Lu1205 were cultured in Dulbecco's modified Eagle's medium (DMEM) supplemented with 10% fetal bovine serum (FBS), and penicillin-streptomycin. The melanoma UACC cell lines (UACC91, UACC502, UACC612, UACC647, and UACC1113) were maintained in Rosewell Park Memorial Institute Medium (RPMI1640) supplemented with 10% FBS and penicillin-streptomycin. In order to develop resistance to B-Raf inhibitor PLX-4032, the cells were grown with increasing concentrations of the inhibitor at each passage and eventually maintained in appropriate media containing either 5 μM (for A375 and Lu1205 cells) or 1 μM (for UACC cells) PLX-4032.

Patient Samples and Immunohistochemistry

Tissue specimen were obtained from patients with metastatic melanoma, harboring the V600E mutation in the B-Raf protein, at various stages during therapy with a B-Raf inhibitor or a combination of B-Raf and MEK inhibitors. The biopsies were collected pre-treatment, after 10-14 days of being on treatment and at time of progression if applicable. The collected tissue samples were formalin fixed and stained with primary antibodies against RNF125 followed by secondary antibody for horseradish peroxidase and subsequently with the chromagen 3,3′-diaminobenzidine (DAB).

Gene Knockdown

Lentiviral pLKO.1 vectors, harboring shRNAs against RNF125, JAK1, and EGFR were transduced into the melanoma cells being tested and silencing efficiency was validated from cell lysates of stable clones using suitable antibodies for total protein content or by qPCR for total mRNA.

Immunoprecipitation

HEK293T cells were transformed with suitable expression vectors, to ectopically express Flag-tagged RNF125 or V5-tagged JAK1. Cell lysates were prepared using a lysis buffer containing 50 mM Tris-HCl, pH 7.4, 150 mM NaCl and 1% TritonX supplemented with protease and phosphatase inhibitors. The lysates were pre-cleared with Protein A/G agarose beads and incubated with appropriate antibodies overnight at 4° C., followed by addition of agarose beads and incubation at 4° C. for an additional 2 hrs. The beads were recovered, washed with the lysis buffer, boiled in Laemmli buffer and subjected to SDS-PAGE analysis.

Liquid Chromatography-Mass Spectrometry (LC/MS-MS)

Protein samples isolated from melanoma cells were reduced, alkylated, trypsin-digested, and desalted. Post desalting, the samples were resuspended in 125 μL of 0.1% TFA/0.2% acetonitrile in glass sample vials. The mass spectrometry samples were analyzed in a Michron MDLC Paradigm MS4 HPLC system, HTC-PAL autosampler, and LTQ Orbitrap XL mass spectrometer.

Cycloheximide Chase Assay

B-Raf inhibitor resistant melanoma cells A375 and Lu1205 were treated with 50 μg/mL of Cycloheximide for 0, 2, 4, 8, and 12 hrs. Samples were collected at the indicated time points and lysates were prepared. The cell lysates were subjected to Western Blot analysis using antibodies against JAK1.

Results:

Correlation Between RNF125 Expression and B-Raf Inhibitor Resistance in Melanoma Patients

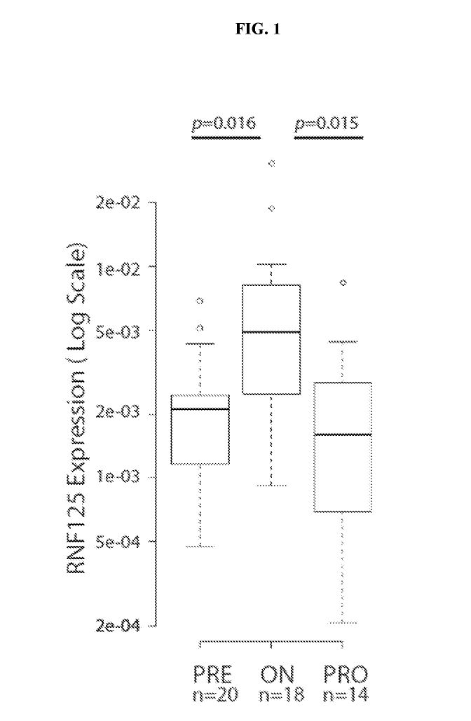

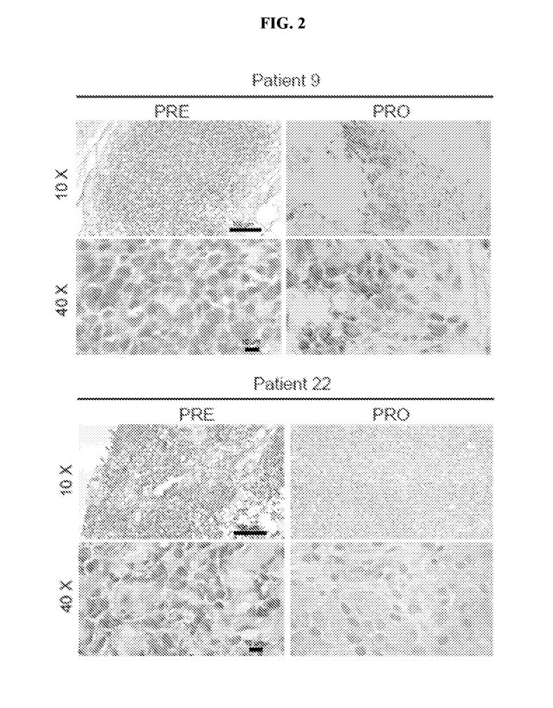

The expression of RNF125 transcript was analyzed in tissue samples collected from melanoma patients, prior to B-Raf inhibitor treatment (PRE), during treatment (ON), and after the development of progressive disease (PRO). As illustrated in FIG. 1 it was found that RNF125 expression in samples from patients treated with B-Raf inhibitor was significantly higher in the initial phase of treatment (ON) but decreased during the progression phase (PRO).

The collected tissue samples were also stained using anti-RNF125 primary antibodies and processed according to methods described for immunohistochemical analysis. The RNF125 expression was found to be reduced in melanoma tissues after disease progression (PRO) relative to the levels prior to B-Raf inhibitor treatment (PRE), as shown in FIG. 2, for tissue samples from two melanoma patients.

Role of Ubiquitin Ligase RNF125 in BRAFi Resistance

Melanoma cell lines UACC91, UACC502, UACC612, UACC647, and UACC1113 were treated with B-Raf inhibitors PLX-4032 or PLX-4720, and the IC50 values were correlated with RNF125 expression levels. The RNF125 expression was determined using RNA-Sequencing (RNA-Seq) methods and expressed in fragments per kilobase per million reads unit (FPKM).

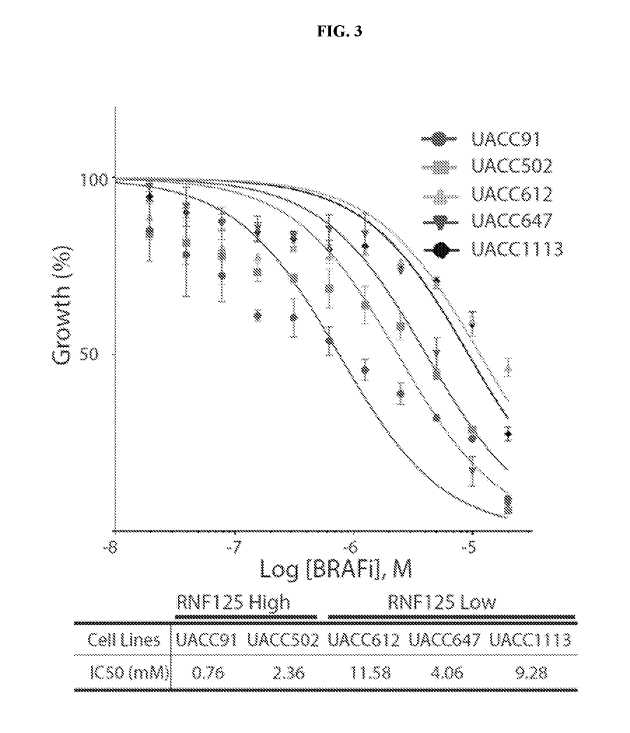

The results, shown in FIG. 3 and Table 1 (for additional cell lines from melanoma patients), revealed an inverse correlation between the RNF125 expression levels and IC50 of the B-Raf inhibitors.

TABLE 1
Inverse Correlation of B-Raf inhibitor resistance
and RNF125 expression in melanoma cell lines

| Melanoma | IC50 to B-Raf Inhibitors | | RNF125 (log |
|---|---|---|---|
| Cell line | PLX-4032 (μM) | PLX-4702 (μM) | 2FPKM + 1) |
| UACC91 | 0.3009 | 0.532 | 11.29 |
| UACC257 | 0.15 | 0.14 | 6.20 |
| UACC502 | 0.55 | 0.78 | 6.90 |
| UACC558 | 0.69 | 0.42 | 4.81 |
| UACC612 | 3.78 | 5.48 | 0.22 |
| UACC647 | 3.37 | 6.72 | 0.22 |
| UACC903 | 0.25 | 0.25 | 1.85 |
| UACC1308 | 0.06 | 0.09 | 11.65 |
| UACC2641 | 5.88 | 4.59 | 0.19 |
| UACC3291 | 0.06 | 0.09 | 6.28 |

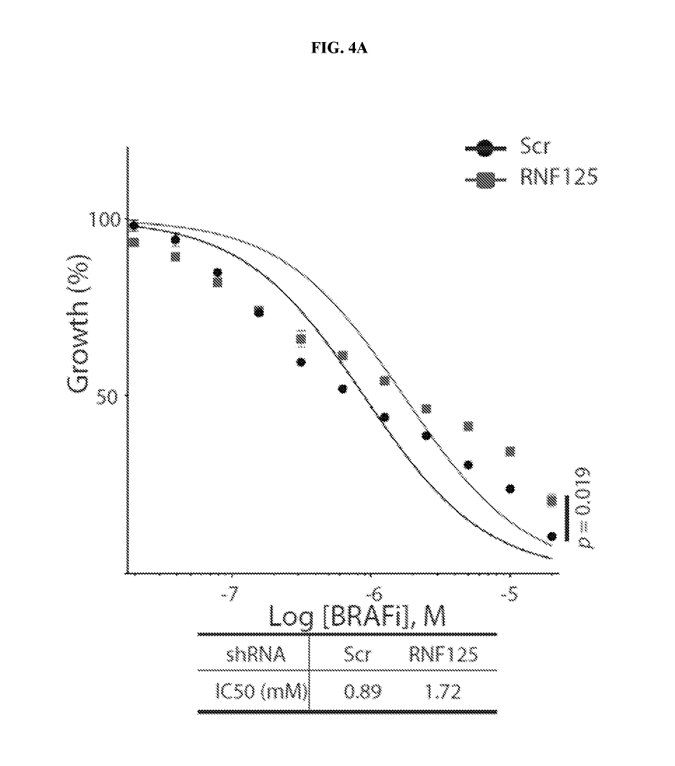

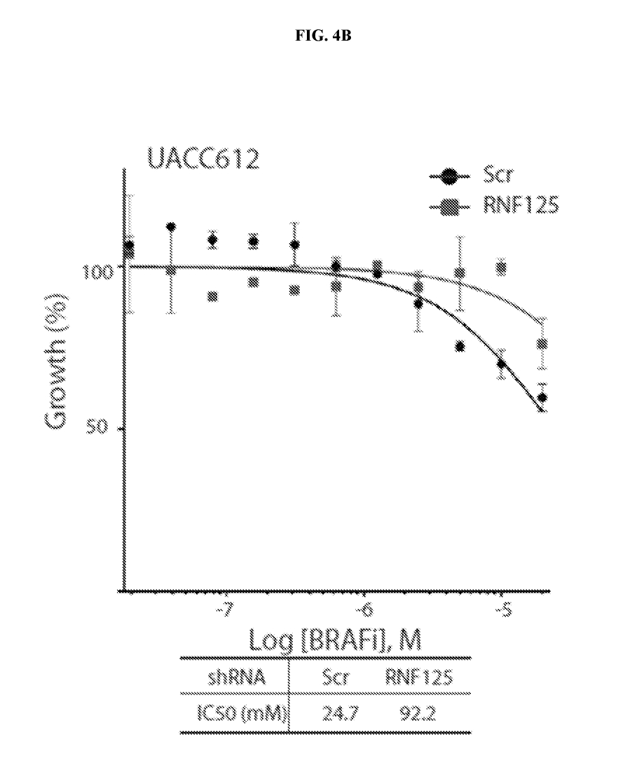

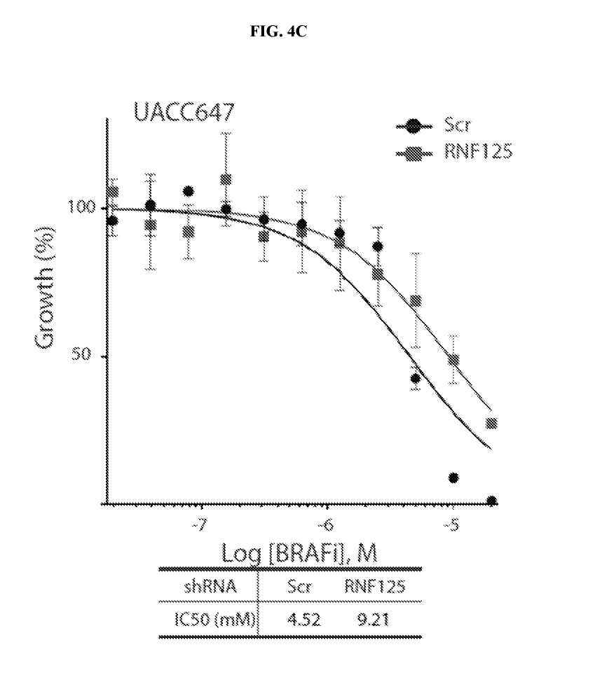

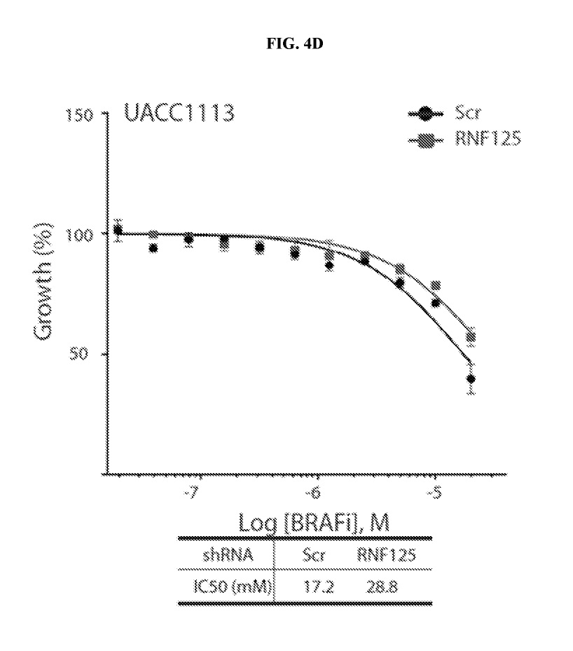

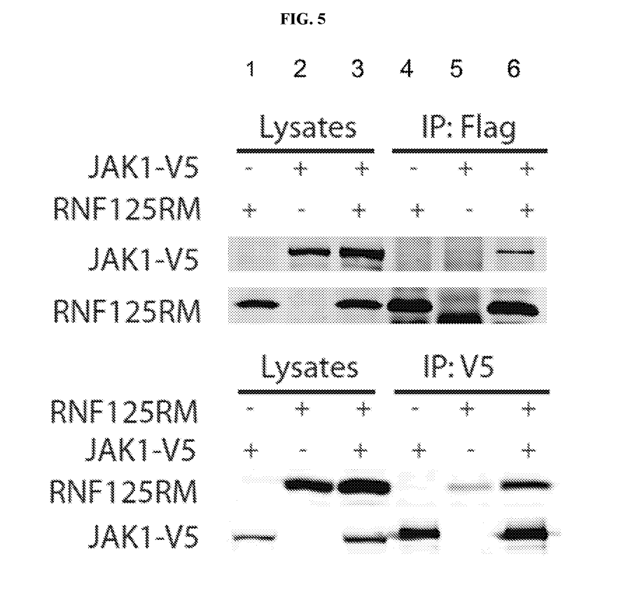

Consistent with the negative correlation, it was observed that when RNF125 was knocked down in various UACC cells, via shRNF125 (small hairpin RNA specific for RNF125), the IC50 to B-Raf inhibitor was increased in those cells, as shown in FIG. 4A-D. The observations established that downregulation of RNF125 expression plays a role in development of resistance to B-Raf inhibitor.

JAK1 as a Substrate for RNF125

JAK1 was identified in a LC-MS/MS screen as a protein that bound to the stably expressed RING mutant form of RNF125, in melanoma cells. The interaction between ectopically expressed JAK1 and RNF125 RING mutant (RNF125RM), in HEK293T cells, was confirmed by immunoprecipitation experiments and the results are shown in FIG. 5. The presence of RNF125RM and JAK1 in the cell lysates were confirmed by immunoblotting with anti-RNF125 or anti-JAK1 antibodies (lanes 1-3). The cell lysates were immunoprecepitated with anti-FLAG or anti-V5 antibodies, as shown in the top and bottom halves, respectively, of FIG. 5. The presence of both RNF125RM and JAK1 in the immunoprocepitated complex (lane 6) demonstrated the interaction between the two proteins.

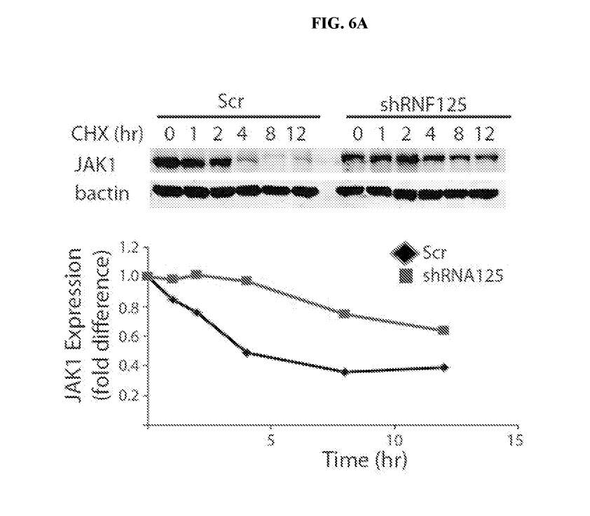

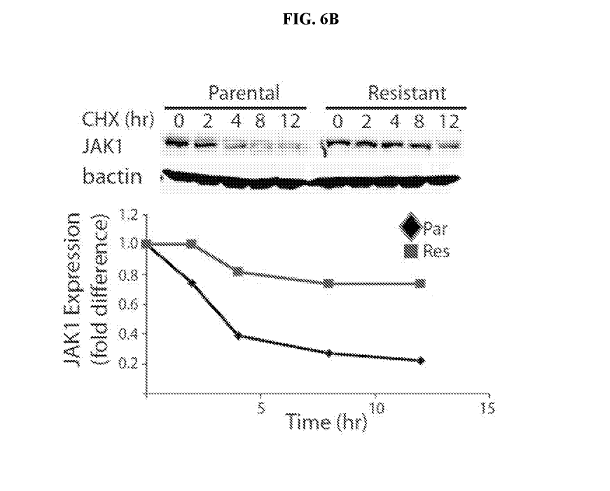

Depletion on RNF125 in A375 cells expressing a scrambled shRNA or shRNF125 resulted in increased expression of JAK1 protein. The cells were subjected to cycloheximide chase, as explained in the Methods section, for the indicated times. Results shown in FIG. 6A, confirmed that lower expression of RNF125 correlated with increased JAK1 levels. Cycloheximide chase analysis was also performed in Lu1205 parental and B-Raf inhibitor resistant cells. Here again, it was seen that JAK1 protein half-life was higher in the resistant cells, characterized by low RNF125 expression, illustrated in FIG. 6B.

Collectively, the results of the immunoprecipitation and the cycloheximide assays, identified JAK1 as a substrate for RNF125.

Post-Transcriptional Regulation of EGFR Levels by RNF125 Through JAK1

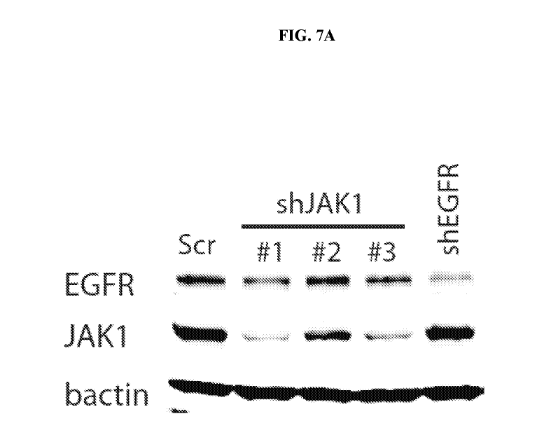

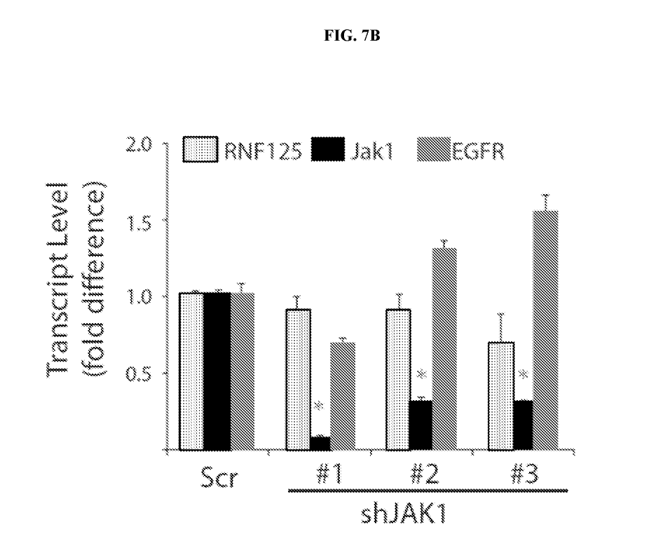

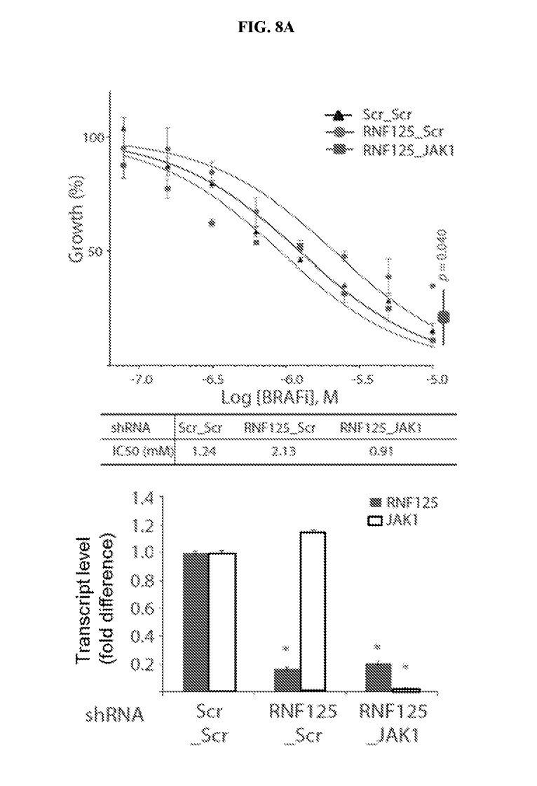

Total protein and transcript levels of EGFR and JAK1 were assessed in UACC melanoma cell lines expressing shRNAs specific towards JAK1 (shJAK1). It was found that depletion of JAK1 in the UACC cells resulted in lowering of EGFR protein levels but the transcript levels of EGFR were not affected. The total protein and transcript levels are shown in FIG. 7A and FIG. 7B, respectively. Based on these results, and the role of RNF125 in regulating JAK1 in the melanoma cell lines, it was concluded that RNF125 post-transcriptionally controlled EGFR expression through JAK1.

Reversal of B-Raf Inhibitor Resistance by JAK1 Depletion, Alone or Together with EGFR

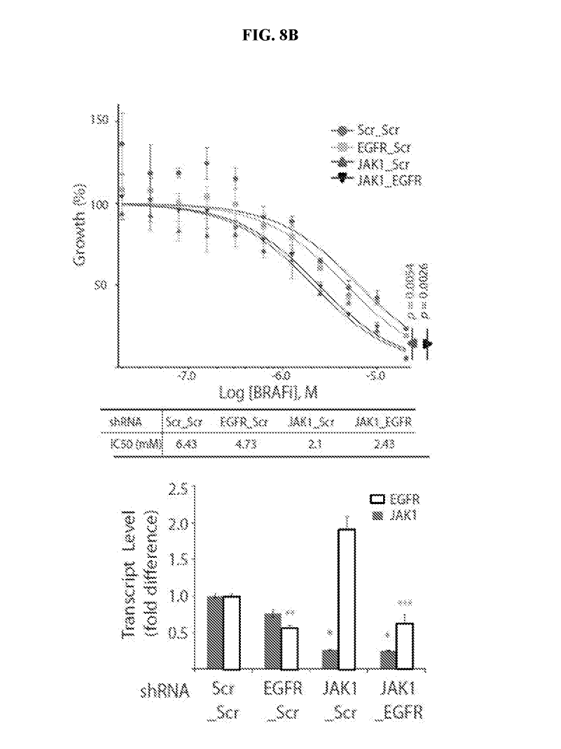

B-Raf inhibitor resistant UACC91 cells were subjected to double knockdown with (a) two scrambled shRNAs, (b) one scrambled shRNA and shRNF125, or (c) shRNF125 and shJAK1. FIG. 8A shows the growth curve and IC50 values for the three different double knockdown UACC91 cells. RNF125 depletion alone resulted in an increase in IC50 from 1.24 μM to 2.13 μM, which could be reduced back to 0.91 μM upon depletion of both RNF125 and JAK1.

Another B-Raf inhibitor resistant cell line UACC1113 was subjected to double knockdown with (a) two scrambled shRNAs, (b) one scrambled shRNA and shEGFR, (c) one scrambled shRNA and shJAK1, (d) shJAK1 and shEGFR. FIG. 8B shows the growth curve and IC50 values UACC1113 cells expressing shRNAs listed above. The basal level of IC50 for these cells was relatively high at 6.43 μM, but the level could be reduced by depletion of JAK1 alone (to 2.1 μM) or in combination with EGFR (2.43 μM). The depletion of EGFR alone did not reduce the IC50 value significantly (4.73 μM).

Inhibition of JAK1 and EGFR Affects Growth of B-Raf Inhibitor Resistant Cells

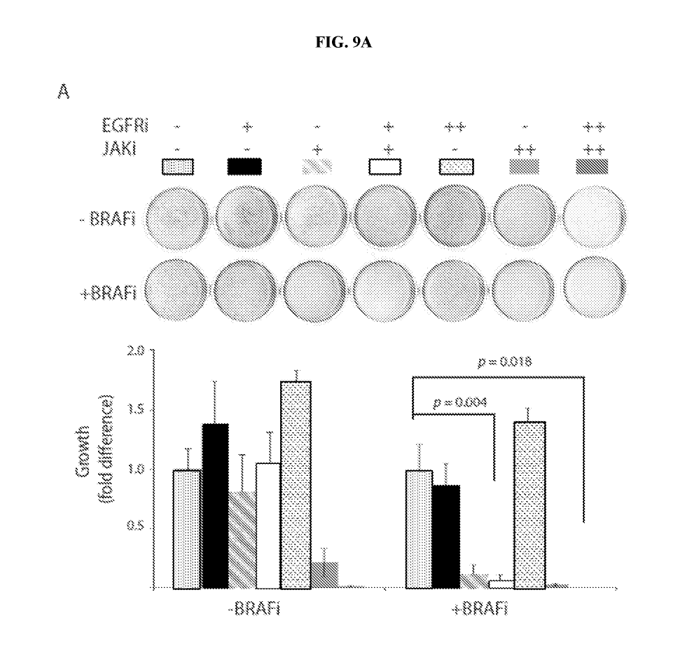

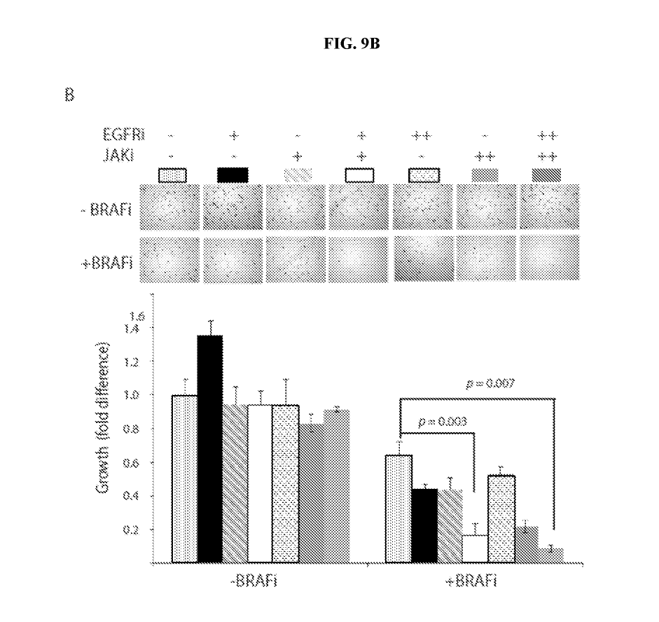

B-Raf inhibitor resistant cells A375 or Lu1205R were grown for two weeks in soft agar, in the presence of B-Raf inhibitor (PLX4032), EGFRi (Gefitinib), and JAKi (Pyridone 6 or AZD1480), in various doses and combinations illustrated in FIG. 9A-B. Single inhibitors or a combination of B-Raf inhibitor and EGFR inhibitor did not inhibit growth in soft agar, however combined treatment with B-Raf inhibitor and JAKi or a triple combination of B-Raf inhibitor, JAK inhibitor, and EGFR inhibitor significantly blocked the growth of the resistant cells, in a dose-dependent manner, as shown in FIG. 9A-B.

### Example 2: Inhibition of JAK1 Arrests Growth of B-Raf Inhibitor and B-Raf Inhibitor and MEK Inhibitor Resistant Melanoma Cells

A375, Lu1205, and several UACC melanoma cell lines that developed resistance to BRAF inhibitors (e.g.—PLX-4032, PLX-4702) or to a combination therapy with BRAF and MEK inhibitors (dabrafenib and trametinib) are selected for the study. The cells are grown for two weeks in soft agar, in the presence of 0-10 μM of ruxolitinib (JAK1 inhibitor), dabrafenib (BRAF inhibitor), trametinib (MEK inhibitor) as single agents or in combination therapies, using a constant volume of inhibitor for all the conditions. Use of any one of the single inhibitors, or a combination of dabrafenib and trametinib only does not block the growth of the melanoma cells. Treatment with a cocktail containing all three inhibitors blocks growth of melanoma cells included in the study.

### Example 3: Combination Therapy with JAK1 Inhibitor and B-Raf Inhibitor and MEK Inhibitor Blocks Tumor Growth in B-Raf Inhibitor and B-Raf Inhibitor and MEK Inhibitor Resistant Mice

Preclinical in vivo efficacy studies are conducted in athymic nude mice. A375 cells are injected subcutaneously in the right lateral flank of the mice. The tumor injected mice are randomly assigned to five groups, each group containing 8-10 animals. Treatment with single or combinations of inhibitors ruxolitinib (JAK1 inhibitor), dabrafenib (B-Raf inhibitor) and trametinib (MEK inhibitor) are initiated once the tumor diameter reaches about 5 mm. Tumor sizes are measured every day with Digital Calipers and tumor volumes are calculated using appropriate computation methods. Tumor growth inhibition is determined by calculating the tumor volume differential between the mice treated with vehicle or the various single or inhibitor combinations described above. Results indicate that tumor growth is slowed down in animals receiving a combination of JAK1, BRAF and MEK inhibitor.

### Example 4: Screening of RNF125 Expression in Melanoma Patients to Select Candidates for Combination Therapy with JAK1, B-Raf, and MEK Inhibitors

The expression of RNF125 protein will be assessed in tumor biopsy specimens collected from melanoma patients harboring mutations (e.g. V600E, V600K) in the B-Raf protein.

In case of patients already being treated with B-Raf inhibitors, or a combination of B-Raf and MEK inhibitors (B-Raf inhibitor and B-Raf and MEK inhibitor resistant patients), tumor biopsies from various stages, before, during and after inhibitor treatment will be stained with anti-RNF125 antibodies and processed using the methods described above for immunohistochemical analysis of patient tissue samples. The stained tissue will be interpreted by a dermatopathologist.

RNF125 transcript levels will also be assessed in cells collected from the melanoma patients, before, during or after treatment with B-Raf inhibitors, alone or in combination with MEK inhibitors.

RNF125 protein and transcript levels will also be assessed in tissue samples and cells collected from melanoma patients who have not been previously treated with B-Raf inhibitor (the B-Raf-inhibitor-naïve patients).

Patients with decreased RNF125 expression upon progression of disease RNF125 will be selected as candidates for combination therapy with JAK1, B-Raf and MEK inhibitors.

### Example 5: Phase 1/2 Study of Ruxolitinib (JAK1 Inhibitor) Combined with Dabrafenib (B-Raf Inhibitor) or Dabrafenib and Trametinib (MEK Inhibitor) in BRAF-Mutant Melanoma Patients Previously Treated with or without B-Raf Inhibitor

Study Population

Patients with any B-Raf inhibitor resistant cancers who meet the study eligibility criteria.

The open label trial will include two patient cohorts. The first cohort will be of BRAF-naïve patients, who have not been treated previously with BRAF or MEK inhibitors. The second cohort will be of B-Raf inhibitor or B-Raf inhibitor and MEKi resistant patients, who have previously been treated with a BRAF inhibitor alone or in combination with a MEK inhibitor.

Phase 1

Primary Outcome

To evaluate the safety and tolerability of ruxolitinib in combination with dabrafenib and trametinib in B-Raf inhibitor-naïve, and B-Raf inhibitor or B-Raf inhibitor and MEK inhibitor resistant patient cohorts.

Phase 2

Primary End-Points:

To document the response rate and duration of response, determined using Response Evaluation Criteria in Solid tumors (RECIST 1.1), of ruxolitinib administered alone or in combination with dabrafenib and trametinib in B-Raf inhibitor-naïve, and B-Raf inhibitor or B-Raf inhibitor and MEK inhibitor resistant melanoma patients.

Secondary End-Points:

To compare the progression-free survival (PFS) of ruxolitinib administered alone vs. in combination with dabrafenib and trametinib in B-Raf inhibitor-naïve, and B-Raf inhibitor or B-Raf inhibitor and MEK inhibitor resistant melanoma patients.

To compare oversall survival rate (OS) of ruxolitinib administered alone vs. in combination with dabrafenib and trametinib in B-Raf inhibitor-naïve, and B-Raf inhibitor or B-Raf inhibitor and MEK inhibitor resistant melanoma patients.

To compare the intra-patient toxicity profile of induction therapy to JAKi maintenance in B-Raf inhibitor-naïve, and B-Raf inhibitor or B-Raf inhibitor and MEK inhibitor resistant melanoma patients treated with ruxolitinib alone or in combination with dabrafenib and trametinib.

To identify suitable biomarkers that can be used for identification and stratification of B-Raf inhibitor-naïve, and B-Raf inhibitor or B-Raf inhibitor and MEK inhibitor resistant melanoma patients for treatment with JAK inhibitor.

To define, the effects of ruxolitinib alone or in combination with dabrafenib and trametinib on the tumor microenvironment, especially on the expression levels of PD-L1 and PD-L2.

Exploratory:

To assess the efficacy of newly developed JAK1 inhibitor (13110) in B-Raf inhibitor-naïve, and B-Raf inhibitor or B-Raf inhibitor and MEKi resistant melanoma cell lines and in syngeneic mouse models.

To assess the possible use of JAK1 inhibitor in other tumors that are considered MAPK pathway dependent.

Study Design and Treatment

A Simon two-stage design, using a power of 80% and a type 1 error rate of 0.1%, will be followed for both B-Raf inhibitor-naïve and B-Raf inhibitor or B-Raf inhibitor and MEK inhibitor resistant patient cohorts.

All patients enrolled in the Phase 1 trial will receive ruxolitinib for a week. Following one week of standard dose of ruxolitinib (10 or 20 mg twice daily), the patients will receive 50% of the standard dose of ruxolitinib combined with standard dose of dabrafenib (75 or 150 mg twice daily) and trametinib (1, 1.5, or 2 mg twice daily), as part of an accelerated Phase 1 trial. If the above mentioned dosage results in zero or one dose limiting toxicity (DLT) case, then the patients will be administered standard doses of ruxolitinib, dabrafenib, and trametinib. If under the full dose regimen, zero or one DLT cases are observed, then three more patients will be enrolled to the Phase 1 trial.

Tumor biopsy specimens will be obtained from all enrolled patients at the baseline stage (before starting ruxolitinib), after one week of administering ruxolitinib, and after one week of combination therapy. The tissue samples will be used for assessment of biomarkers of B-Raf inhibitor resistance and response to ruxolitnib alone or in combination with dabrafenib and trametinib. Assessments may include but not limited to evaluation of expression of RNF125, JAK1, EGFR, AXL, PDL-1/2, pERK, SOX10/MITF. The pERK expression levels may be evaluated using the Terminal deoxynucleotidyl transferase (TUNEL) assay.

Statistical Analysis

The null hypothesis for the B-Raf inhibitor or B-Raf inhibitor and MEK inhibitor resistant patient cohort will assume a response rate of 10% or less, and a response greater than 20% will be considered as an adequate outcome for enrolling more patients in the cohort. The Phase 2 study for this cohort will begin by enrolling 14 patients. If one patient shows a response of 20% or higher, the study will be expanded to include an additional 22 patients.

In case of the B-Raf inhibitor-naïve patient cohort a response rate greater than 85% will be considered of interest. In addition, the response duration should also be maintained for 6 months or longer. The Phase 2 study for this cohort will begin by accruing 20 patients. No new patient will be enrolled until the 20^{th} patient completes the 8 month follow-up, a time point at which the response rate and duration of response will be calculated. If the results satisfy the efficacy threshold that is response rate >85% and response duration >6 months, then 21 more patients would be accrued.

Tumor biopsy specimens will be collected periodically during the Phase 2 study and used for assessing changes in biomarkers, as described earlier, due to treatment with ruxolitinib alone or in combination with dabrafenib and trametinib.

Statistical Analysis for Secondary Endpoints

The secondary endpoints of PFS and OS will be assessed using the Kaplan-Meier method. The overall response rate (ORR) within each patient cohort will be estimated by the number of subjects with either complete or partial remission (CR or PR) with 90% confidence intervals, using RECIST 1.1.

Safety Review

All reported adverse events will be summarized as part of a safety review at the end of the first stage. The list of adverse events that may be monitored includes, but are not limited to, pyrexia, chills, fatigue, nausea, vomiting, diarrhea, headache, peripheral edema, cough, arthralgia, rash, night sweats, decreased appetite, myalgia, constipation, elevated blood alkaline phosphatase, hypertension, decreased cardiac ejection fraction, ocular events, hyperproliferative skin lesions (e.g.—cutaneous squamous-cell carcinoma, papilloma, and hyperkeratosis) etc.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating cancer in an individual in need thereof comprising administration of a JAK inhibitor and an inhibitor targeting the MAPK pathway to the individual.

2. The method of claim 1, wherein the JAK inhibitor is a JAK1 inhibitor.

3. The method of claim 1, wherein the JAK inhibitor is a compound selected from

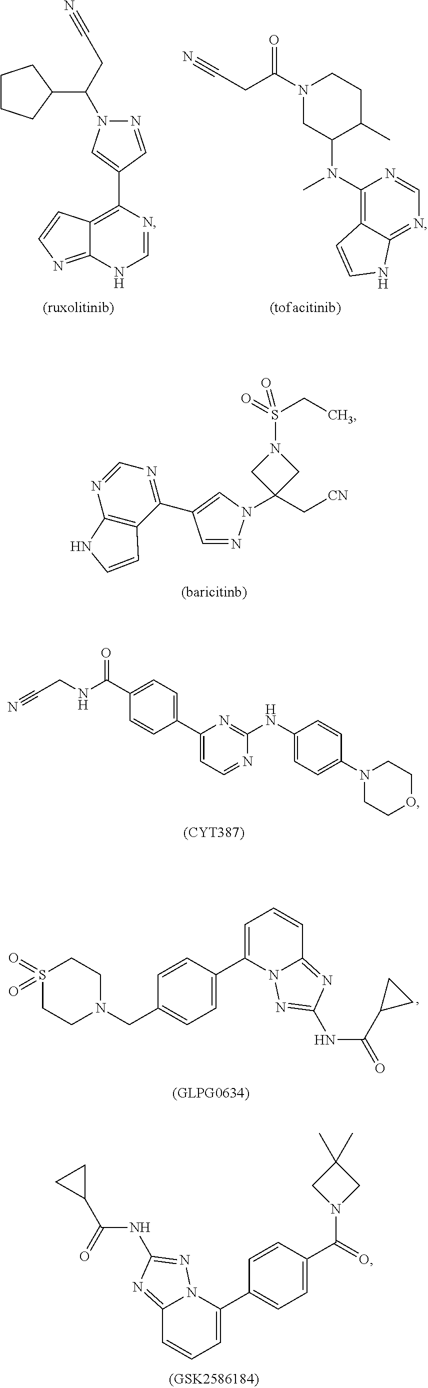

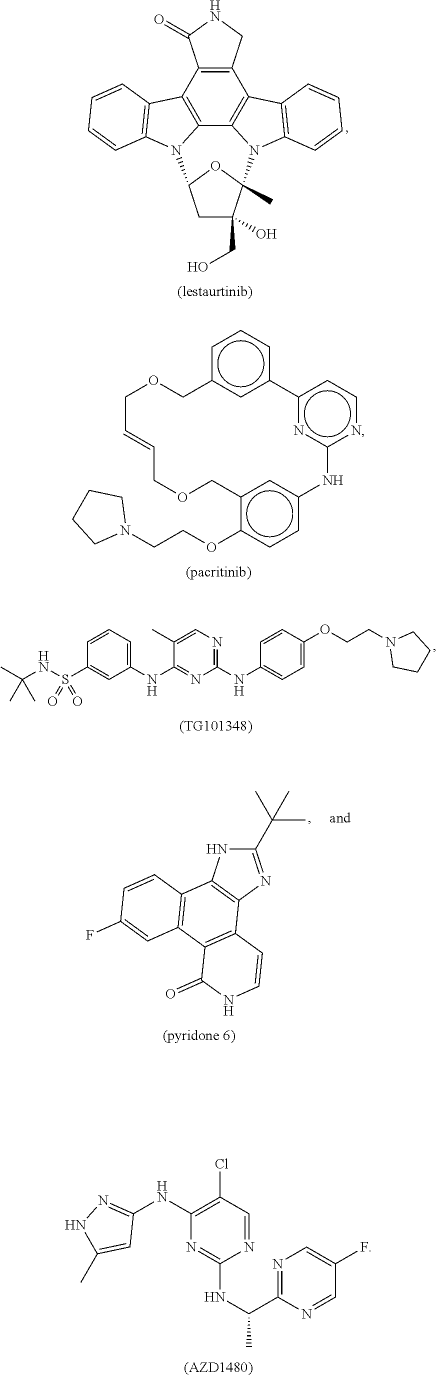

4. The method of claim 1, wherein the JAK inhibitor is ruxolitinib.

5. The method of claim 1, wherein the inhibitor targeting the MAPK pathway is a B-Raf inhibitor.

6. The method of claim 5, wherein the B-Raf inhibitor is selected from

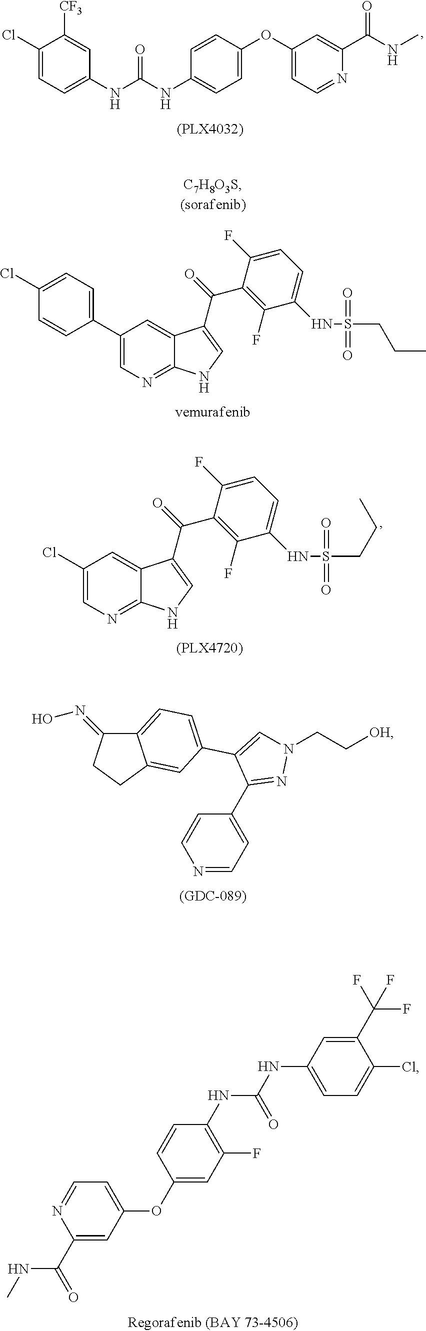

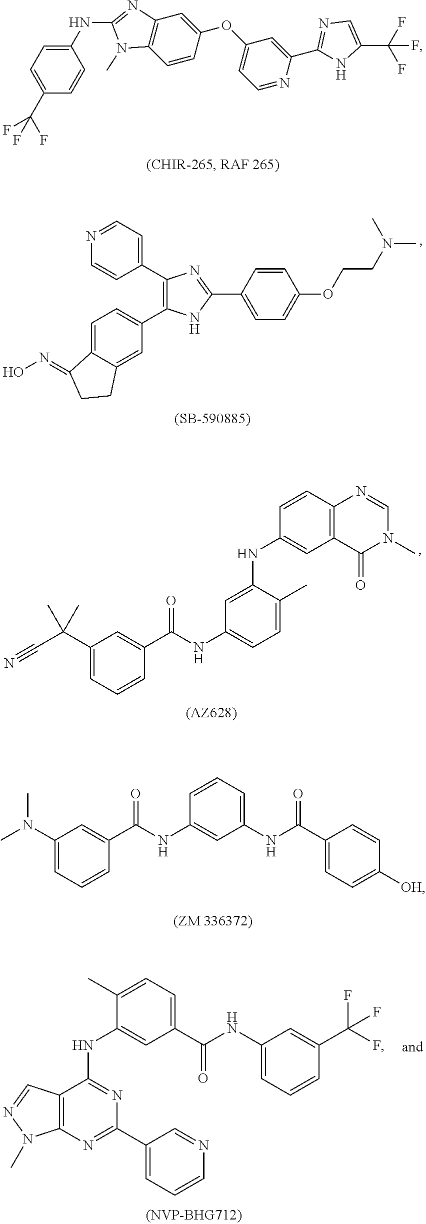

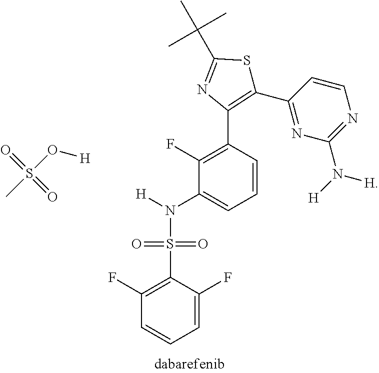

7. The method of claim 1, wherein the B-Raf inhibitor is dabarefenib.

8. The method of claim 1, wherein the B-Raf inhibitor is vemurafenib.

9. The method of claim 1, wherein the cancer is a BRAF mutant cancer.

10. The method of claim 1, wherein the cancer is selected from the group consisting of head and neck cancer, lung cancer, ovarian cancer, colon cancer, breast cancer, pancreatic cancer, cervical cancer, prostate cancer, and melanoma.

11. The method of claim 1, wherein the cancer is melanoma.

12. The method of claim 1, wherein the cancer is B-Raf inhibitor resistant melanoma.

13. The method of any of claims 1-12, wherein the inhibitor targeting the MAPK pathway is a MEK inhibitor.

14. The method of claim 13, wherein the MEK inhibitor is selected from the group consisting of

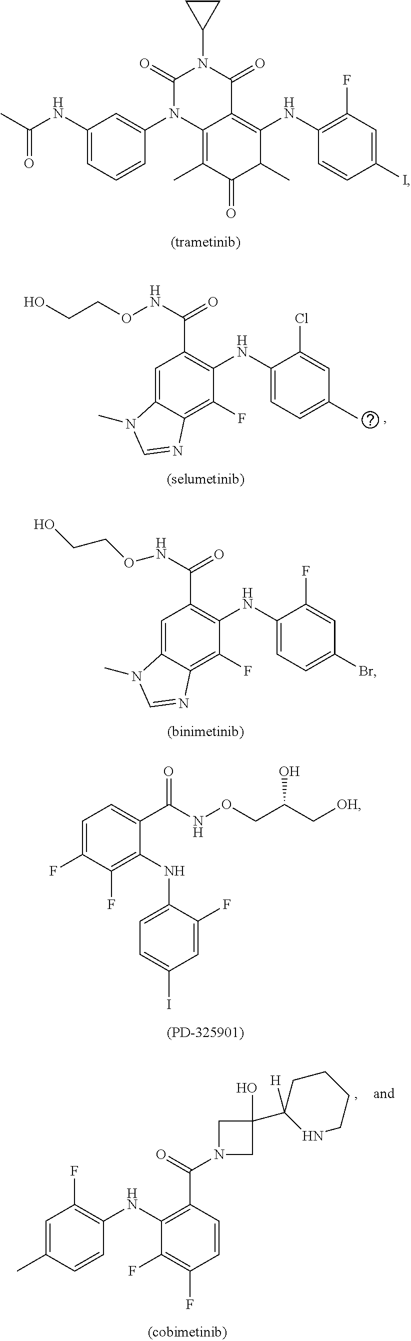

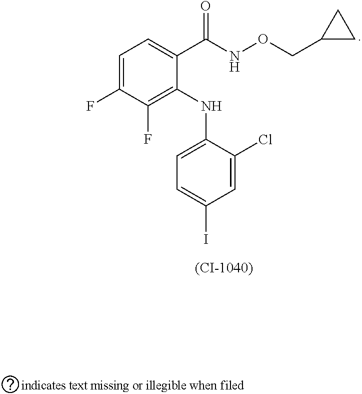 [SMILES: O=C(NOCC1CC1)c1ccc(F)c(F)c1Nc1ccc(I)cc1Cl]

15. The method of claim 12, wherein the MEK inhibitor is trametinib.

16. The method of any of claims 1-15, wherein the inhibitor targeting the MAPK pathway is an ERK1/2 inhibitor.

17. The method of claim 16, wherein the ERK inhibitor is SCH772984 or VTX11e.

18. The method of any of claims 1-17, wherein the method further comprises determining the level of RNF125 in the individual.

19. The method of any of claims 1-17, wherein the method further comprises determining the level of JAK1 in the individual.

20. The method of any of claims 1-17, wherein the method further comprises determining the level of EGFR in the individual.

21. The method of any of claims 1-17, wherein the method further comprises determining the level of AXL in the individual.

22. A method for treating cancer in an individual in need thereof comprising administration of a JAK inhibitor, a B-Raf inhibitor and a MEK inhibitor to the individual.

23. A method for treating cancer in an individual in need thereof comprising administration of a JAK inhibitor, a B-Raf inhibitor and an ERK1/2 inhibitor to the individual.

24. A method for treating cancer in an individual in need thereof comprising administration of a JAK inhibitor, a MEK inhibitor and an ERK1/2 inhibitor to the individual.

25. A method for treating cancer in an individual in need thereof comprising administration of a JAK inhibitor, a B-Raf inhibitor, a MEK inhibitor and an ERK1/2 inhibitor to the individual.

26. A method for treating a BRAF mutant cancer in an individual in need thereof comprising administration of ruxolitinib and dabrafenib to the individual in need thereof.

27. A method for treating a BRAF mutant cancer in an individual in need thereof comprising administration of ruxolitinib, dabrafenib and trametinib to the individual in need thereof.

28. A method for treating cancer in an individual in need thereof comprising:
determining the level of RNF125 activity or expression in the cancer cells of an individual, and administering to the individual a JAK inhibitor and an inhibitor targeting the MAPK pathway to the individual if the RNF125 activity or expression is reduced in the cancer cells.

29. A method of monitoring and treating an individual with a BRAF mutant cancer for resistance to a B-Raf inhibitor comprising:
determining the level of RNF125 activity or expression in the cancer cells of an individual on a periodic basis, and administering to the individual a JAK inhibitor in combination with an inhibitor targeting the MAPK pathway if the RNF125 activity or expression is reduced in the cancer cells.

30. A method of identifying and treating an individual with a BRAF mutant cancer for resistance to a B-Raf inhibitor comprising:
determining the level of RNF125 activity or expression in the cancer cells of an individual;
identifying the individual as being resistant to a B-Raf inhibitor if the RNF125 activity or expression is low in the cancer cells; and administering to the individual a JAK inhibitor and an inhibitor targeting the MAPK pathway to the individual if the RNF125 activity or expression is reduced in the cancer cells.

31. A method for treating cancer in an individual having reduced RNF125 activity or expression comprising administration of a JAK inhibitor and an inhibitor targeting the MAPK pathway to the individual.

32. The method of any one of claims 28 to 31, wherein the inhibitor targeting the MAPK pathway is a B-Raf inhibitor.

33. The method of any one of claims 28 to 31, wherein the inhibitor targeting the MAPK pathway is a MEK inhibitor.

34. The method of any one of claims 28 to 31, wherein the inhibitor targeting the MAPK pathway is an ERK1/2 inhibitor.
